(19) United States
(12) Patent Application Publication
(10) Pub. No.: US 2024/0108682 A1
(43) Pub. Date: Apr. 4, 2024

(54) COMPOSITIONS AND METHODS FOR INCREASING GLUTATHIONE LEVELS

(71) Applicant: Lile Method Research, LLC, Franklin, TN (US)

(72) Inventor: Laura Lile, Franklin, TN (US)

(73) Assignee: Lile Method Research, LLC, Franklin, TN (US)

(21) Appl. No.: 18/370,919

(22) Filed: Sep. 21, 2023

Related U.S. Application Data
(60) Provisional application No. 63/457,919, filed on Apr. 7, 2023.
(60) Provisional application No. 63/383,130, filed on Nov. 10, 2022.
(60) Provisional application No. 63/376,591, filed on Sep. 21, 2022.

(51) Int. Cl. A61K 38/01; A61K 31/198; A61K 33/04; A61K 33/22

(52) U.S. Cl. CPC A61K 38/014; A61K 31/198; A61K 33/04; A61K 33/22

(57) ABSTRACT

Glutathione support compositions and methods for increasing glutathione levels, especially intracellular glutathione levels, and/or methods for improving vaccine therapy and reducing gamma-glutamyltransferase (GGT) levels in an individual. In addition, the disclosure describes methods for boosting immunity, treating and preventing infectious diseases such as tuberculosis and MRSA, and combating the effects of aging and age-related stress, oxidative stress, and inflammation. The glutathione support compositions include a collagen source, a glutamate source, a cysteine source, and a selenium source, and, optionally, a boron source.

### CROSS-REFERENCE TO RELATED APPLICATIONS

This application claims priority to U.S. Provisional Patent Application No. 63/376,591, filed Sep. 21, 2022, U.S. Provisional Patent Application No. 63/383,130, filed Nov. 10, 2022, and U.S. Provisional Patent Application No. 63/457,919, filed Apr. 7, 2023, the specification, drawings, and appendices thereof are incorporated herein by reference in their entirety.

### FIELD OF THE INVENTION

The present disclosure relates to compositions and methods for increasing glutathione levels, especially intracellular glutathione levels, treating and preventing infections and diseases, improving vaccine therapy, and combating aging and age-related cellular damage. In particular, the compositions of the present disclosure may be used to prevent and treat tuberculosis and MRSA in mammals through increased intracellular levels of glutathione in a subject. In addition, the compositions of the present disclosure may be used to improve vaccine therapy by mitigating or preventing intracellular damage and unwanted intracellular and systemic inflammation, avoiding anti-body dependent enhancement, and neutralizing damaging free radicals. Moreover, the compositions of the present disclosure may be used to reduce gamma-glutamyltransferase (GGT) levels in the blood.

### BACKGROUND OF THE INVENTION

Oxidative stress arises when increasing levels of reactive oxygen species (ROS) overwhelm the activity of antioxidant enzymes, including catalase, superoxide dismutase (SOD), or glutathione peroxidase (GSH-PX). ROS act as second messengers in many intracellular signaling cascades and lead to cellular macromolecular damage, such as membrane lipid breakdown, DNA fragmentation, protein denaturation and mitochondrial dysfunction, which greatly influence cell metabolism and signaling. In other words, oxidative stress is a deleterious process that can damage cell structures, including lipids, proteins, and DNA.

There is a link between oxidative stress and nearly all diseases and maladies. For example, unchecked oxidative stress can cause chronic inflammation, which plays a role in aging, immunity, overall health, including mental health, and disease. Inflammation is associated with most chronic diseases such as cancer, diabetes, cardiovascular, neurological, and pulmonary diseases.

Glutathione—a tripeptide formed from three amino acids (cysteine, glycine, and glutamine or glutamic acid)—acts as an important antioxidant, which helps protect the body from damage to cells caused by free radicals. It is essential for proper functioning of the immune system and is vital for building and repairing tissue. Under normal physiological conditions, glutathione is present in cells in relatively high concentrations. See, e.g., van't Erve, Thomas J. et al., The concentration of glutathione in human erythrocytes is a heritable trait, FREE RADIO BIOL MED., 65:742-749 (2013) (summarizing glutathione levels reported in the literature and providing an estimated intracellular glutathione concentration range of 0.4 to 3.0 mM (mean 1.4 mM)). Glutathione exists in cells in two states: reduced (GSH) and oxidized (GSSG). Healthy cells at rest have a GSH/GSSG ratio >100 while the ratio drops to 1 to 10 in cells exposed to oxidative stress.

Glutathione plays a central role in shielding cellular macromolecules from endogenous and exogenous reactive oxygen and nitrogen species. Therefore, maintaining or enhancing intracellular glutathione allows the body to naturally fend off cellular damage. However, improving intracellular glutathione levels is challenging. Indeed, supplying glutathione directly to cells is not a viable option, for several reasons, including the fact that glutathione synthesis is subject to negative feedback inhibition, such that supplying glutathione to cells can halt native glutathione synthesis. Ballatori N. et al., Glutathione dysregulation and the etiology and progression of human diseases, BIOL CHEM. 390(3):191-214 (2009). In addition, the half-life of glutathione in the blood is only seconds to minutes. Lu SC, Regulation of glutathione synthesis, CURR TOP CELL REGUL. 36:95-116 (2000). Furthermore, any glutathione that remains in the blood must overcome thermodynamic and biochemical hurdles to enter the cell since as glutathione is membrane-impermeable.

Thus, there remains a need for safe, effective, and simple treatments to reliably increase glutathione levels. The compositions and methods of the instant disclosure address this need and, in doing so, also address other needs. For example, tuberculosis (TB), which is a chronic infectious disease caused by infection with Mycobacterium tuberculosis and other Mycobacterium species that is a major pandemic disease in developing countries, as well as an increasing problem in developed areas of the world, can generally be controlled using extended antibiotic therapy, but such treatment is not sufficient to prevent the spread of the disease. More specifically, current clinical practice for latent tuberculosis (asymptomatic and non-contagious) is treatment with 6 to 9 months of isoniazid or other antibiotic or alternatively 4 months of rifampin. Active tuberculosis is treated with a combination of four medications for 6 to 8 weeks during which the majority of bacilli are thought to be killed, followed by two drugs for a total duration of 6 to 9 months. Similarly, methicillin-resistant Staphylococcus aureus (MRSA) infection (including health care-associated MRSA (HA-MRSA) and community-associated MRSA (CA-MRSA)) is caused by a type of staph bacteria that has become resistant to many antibiotics used to treat ordinary staph infections. MRSA infections usually require intravenous antibiotics, sometimes for long periods of time depending on the severity of the infection. Methods for preventing infection and improvements with the current treatments for TB and MRSA are needed. The compositions and methods of the present disclosure address this need.

Likewise, while conventional vaccines are an effective and critical tool for controlling infectious diseases, they have limitations and there is a need to improve such therapies. For example, conventional vaccines require extensive time and materials for production, creating difficulty for large-scale deployment. They also rely on the adaptive instead of the innate immune response, which some infections can evade. And, the newer vaccine therapies that rely on messenger RNA (mRNA), viral vector, and recombinant subunit vaccines, a major challenge in developing such new therapies is increasing effective immune activation, leading to protective responses, while limiting unwanted inflammation, toxicity, and intracellular damage. Thus, providing effective protection while limiting side effects is critical to successful vaccine therapies. The compositions and methods of the present disclosure address this need.

In addition, there is a need to decrease gamma-glutamyltransferase (GGT) levels in hepatocytes so as to allow for better liver function and quicker self-repair. More particularly, when the liver is under stress, such is the case with liver disease or damage, GGT levels may increase as the liver attempts to compensate for damage. Low antioxidant defenses are also correlated with elevated GGT. GGT induces oxidative stress in the artery wall in the presence of free iron, and GGT also likely is an indicator of depleted supply of glutathione, especially in the liver, which leads to a cascade of problems related to increased oxidative stress. As a result, there is a need for a solution that allows a body to continuously synthesize glutathione so that the liver is better equipped to combat oxidative stress and other forms of damage. The compositions of the present disclosure address this need.

### SUMMARY OF THE INVENTION

The present disclosure is drawn to glutathione support compositions and methods for increasing glutathione levels, especially intracellular glutathione levels. In addition, the compositions and methods are useful for boosting immunity, treating, and preventing infectious diseases, combating against potential harm from exposure to ionizing radiation, post traumatic brain injury and combating the effects of aging and age-related stress and deterioration of the body. The compositions and methods are based, in part, on the discovery that a formulation including a collagen source including protein or peptides containing a high quantity of proline residues, hydroxyproline residues, and glycine residues with a glutamate source, a cysteine source, a selenium source, and, optionally, boron, is surprisingly effective for potentiating glutathione synthesis and providing additional health and anti-aging benefits.

The glutathione support composition includes a collagen source, a glutamate source, a cysteine source, a selenium source, and, optionally, boron. The collagen source may be provided by the protein or peptides having a high concentration of glycine residues. In some embodiments, the collagen source includes peptides having at least 20 weight percent glycine residues (based on the total average weight of the proteins or peptides). Sources of glutamate and cysteine are also included in the compositions. In some embodiments, the glutamate source includes L-glutamine, an L-glutamine precursor, a salt thereof, or a combination thereof. In other embodiments, the cysteine source incudes L-cystine, an L-cysteine precursor, a salt thereof, or a combination thereof. A selenium source is also included in the formulation. Boron may also be included in the formulation.

In some embodiments, the glutathione support compositions include:
(a) a collagen source that includes proteins or peptides including at least 5 percent proline residues, at least 5 percent hydroxyproline residues, and at least 20 percent glycine residues, based on a total weight average of the proteins or peptides;
(b) a glutamate source such as L-glutamine, an L-glutamine precursor, a salt thereof, or a combination thereof;
(c) a cysteine source such as L-cystine, an L-cysteine precursor other than L-cystine, a salt thereof, or a combination thereof;
(d) a selenium source; and
(e) optionally, a boron source and/or one or more additional active ingredients.

In some embodiments, the collagen source includes collagen, hydrolyzed collagen, gelatin, hydrolyzed gelatin, elastin, hydrolyzed elastin, glycine-rich RNA-binding proteins, tenascin-C, proteoglycans, hydrolysates thereof (peptides thereof), or a combination thereof. In various embodiments, hydrolyzed collagen, hydrolyzed gelatin, or a mixture thereof is preferred. The collagen, hydrolyzed collagen, gelatin, hydrolyzed gelatin, or mixture thereof may include or be selected from Type I, Type II, Type III, Type IV, Type V, or a combination thereof in various embodiments, the collagen, hydrolyzed collagen, gelatin, hydrolyzed gelatin, or mixture thereof comprises or consists of Type I and optionally Type III.

In other embodiments, L-glutamine precursors include esters of L-glutamine, glutamic acid, esters of glutamic acid, glutamine dipeptides (for example glutamine-glutamine dipeptide, glycyl-L-glutamine dipeptide, etc.) salts thereof, or a combination thereof. In other embodiments, salts of glutamic acid include monosodium glutamate and monopotassium glutamate are the glutamate source. In still other embodiments, the glutamate source is an ester of L-glutamine such as L-glutamine methyl ester and L-glutamine methyl ester hydrochloride, L-glutamine t-butyl ester, and L-glutamine t-butyl ester hydrochloride. In yet other embodiments, esters of glutamic acid include L-glutamic acid 1-methyl esters, L-glutamic acid dimethyl ester, and L-glutamic acid 5-ethyl ester are used as the glutamate source. In other embodiments, the glutamate source is a L-glutamine dipeptide such as L-glycyl-L-glutamine (Gly-Gln), L-arginyl-L-glutamine (Arg-Gln) and L-alanyl-L-glutamine (Ala-Gln).

In still other embodiments, the cysteine source includes L-cystine, N-acetyl cysteine, L-cysteine esters, for example, L-cysteine methyl ester or L-cysteine ethyl ester, salts thereof, anhydrides thereof, or combinations thereof.

In yet other embodiments, the selenium source includes selenomethionine, selenocysteine, selenite, methyl selenocysteine, selenium nanoparticles, selenocysteine, Se-methyl selenocysteine, or combinations thereof.

In other embodiments, the boron source includes salts of boron, for example, sodium borate, which is also known as sodium tetraborate or disodium tetraborate), boron amino acid chelate, boron ascorbate, boron aspartate, boron citrate, boron gluconate, boron glycinate, boron picolinate, calcium fructoborate, or combinations thereof.

In still other embodiments, the glutathione support compositions include an additional active ingredient (or it can replace the boron source). In this aspect, the additional active ingredient includes a zinc source, methylfolate, methyl-B₁₂, quercetin, sulforaphane, methionine, vitamin D, vitamin C, L-threonine, L-theanine, taurine, curcumin, turmeric, melatonin, ashwagandha, fisetin, 1-NMA, NAD+, or kaempferol (or other flavonoid antioxidants), or combinations thereof.

In yet other embodiments, the glutathione support compositions includes one or more carriers and/or fillers.

The present disclosure also relates to glutathione support compositions that includes about 50 to about 99.99 weight percent, 60 to about 99.99 weight percent, or about 75 to about 99.99 weight percent of a combination of (a), (b), and (c):
(a) a collagen source;
(b) a glutamine source; and
(c) a cysteine source (relative to a total weight of the glutathione support composition).

In some embodiments, the glutathione support composition includes about 30 to about 80 weight percent (based on the total weight of the glutathione support composition) of (a), based on the total weight of the glutathione support composition. In other embodiments, the glutathione support composition includes about 10 to about 50 weight percent of (b) (based on the total weight of the glutathione support composition). In yet other embodiments, the glutathione support composition includes about 5 to about 30 weight percent of (c) (based on the total weight of the glutathione support composition).

In some aspects, the glutathione support compositions can be in a variety of different forms. In one embodiment, the compositions are in the form of a powder, a liquid, a tablet, a capsule, an edible, a gummy, a beverage, a carbonated beverage, in a sachet, or a chewable or effervescent tablet.

In other aspects, the glutathione support compositions are useful for increasing glutathione levels, especially intracellular glutathione levels in mammals, particularly humans. In addition, the glutathione support compositions are useful in methods for boosting immunity, treating and preventing infectious diseases, and combating the effects of aging and age-related stress and deterioration of the body. In still other aspects, the glutathione support compositions are useful for improving vaccine therapy by increasing intracellular glutathione levels in an individual while the individual develops vaccine-induced immunity. Additional benefits, embodiments, and methods for using the glutathione support compositions are discussed below.

The present disclosure also relates to a glutathione support composition for increasing intracellular glutathione levels in a subject, including:
(a) a collagen source, wherein the collagen source includes proteins or peptides including at least 5 percent proline residues, at least 5 percent hydroxyproline residues, and at least 20 percent glycine residues based on a total weight average of the proteins or peptides;
(b) a glutamate source;
(c) a cysteine source; and
(d) a selenium source.
   In some embodiments, the glutathione support also includes a boron source. In other embodiments, the glutathione support composition also includes one or more active ingredients. In some aspects, the one or more active ingredients is selected from the group consisting of a zinc source, methylfolate, methyl-B₁₂, quercetin, sulforaphane, methionine, vitamin D, vitamin C, L-threonine, L-theanine, turmeric, curcumin, taurine, melatonin, ashwagandha, fisetin, 1-NMA, NAD+, or kaempferol (or other flavonoid antioxidants), and combinations thereof. In other aspects, the collagen source is present in an amount of about 30 to about 60 weight percent, the glutamate source is present in an amount of about 20 to about 50 weight percent, and the cysteine source is present in an amount of about 10 to about 30 weight percent, based on the total weight of the glutathione support composition. In still other aspects, the collagen source and the glutamate and cysteine sources are in present in an ratio of 1:3 to 3:1 ((a):(b)+(c)). In yet other aspects, the glycine, glutamate, and cysteine sources ((a)+(b)+(c)) constitute about 50 to about 99.99 weight percent based on the total weight of the glutathione support composition.

In various embodiments, the glutathione support composition ma include collagen, collagen peptides, gelatin, gelatin peptides, elastin, elastin peptides, glycine-rich RNA-binding proteins, tenascin-C, proteoglycans, hydrolysates thereof (peptides thereof), or combinations thereof. In some aspects, the collagen source includes collagen peptides, gelatin peptides, elastin peptides, or a combination thereof. In other aspects, the cysteine source includes L-cystine, N-acetyl cysteine, a salt thereof, or a combination thereof. In still other aspects, the glutamate source includes L-glutamine, a L-glutamine precursor, glutamic acid, a glutamic acid precursor, a salt thereof, or a combination thereof. In yet other aspects, the selenium source is selected from selenomethionine, selenocysteine, selenite, methyl selenocysteine, selenium nanoparticles, selenocysteine, Se-methylselenocysteine, and a combination thereof. For example, the selenium source may be selenomethionine, methylselenocysteine, and combinations thereof.

The present disclosure also relates to a glutathione support composition for increasing intracellular glutathione levels in a subject, including:
(a) a collagen source present in an amount of about 35 to about 75 weight percent based on the total weight of the composition, wherein the collagen source includes hydrolyzed collagen, hydrolyzed gelatin, hydrolyzed elastin, or a mixture thereof;
(b) a glutamate source present in an amount of about 20 to about 50 weight percent based on the total weight of the composition, wherein the glutamate source includes L-glutamine, a L-glutamine precursor, a salt thereof, or a combination thereof;
(c) a cysteine source present in an amount of about 10 to about 30 weight percent based on the total weight of the composition, wherein the cysteine source includes L-cystine, a L-cysteine precursor other than L-cystine, a salt thereof, or a combination thereof; and
(d) a selenium source.

In some embodiments, the selenium source is present in an amount such that the glutathione support composition includes about 50 to about 300 ppm of elemental selenium. In other embodiments, the glutathione support composition also includes a boron source. In some aspects, the boron source is present in an amount such that the glutathione support composition includes about 20 to about 400 ppm of elemental boron. In yet other embodiments, the glutathione support composition may also include taurine, melatonin, sulforaphane, quercetin, Vitamin D3, Vitamin k2, Vitamin C, metformin, L-threonine, L-theanine, turmeric, curcumin, ashwagandha or an extract thereof, fish oil, krill oil, omega 3 fatty acids, odd-chain fatty acids, botanical extracts, or a combination thereof.

The present disclosure also relates to a method of increasing intracellular glutathione in a subject, including:
administering a glutathione support composition to the subject in a therapeutically effective amount, wherein the glutathione support composition includes:
   (a) a collagen source, wherein the collagen source includes proteins or peptides including at least 5 percent proline residues, at least 5 percent hydroxyproline residues, and at least 20 percent glycine residues based on a total weight average of the proteins or peptides;
   (b) a glutamate source;
   (c) a cysteine source;
   (d) a selenium source; and
   (e) a boron source.
      In some embodiments, the step of administering includes providing the subject the therapeutically effective amount in a daily dosage, wherein the daily dosage is administered to the individual for a dosing period of at least 3 consecutive days, and wherein the subject has a ratio of intracellular reduced glutathione (GSH) to oxidized glutathione (GSSH) of at least 5:1 after the dosing period. In other embodiments, the collagen source is present in an amount of about 35 to about 75 weight percent based on the total weight of the glutathione support composition, and wherein the collagen source includes hydrolyzed marine or ovine collagen, hydrolyzed marine or ovine gelatin, hydrolyzed elastin, or a mixture thereof.

The present disclosure further relates to a method for reducing gamma-glutamyltransferase (GGT) levels in a subject, including:
administering to the subject a therapeutically effective amount of a glutathione support composition including:
   (a) a collagen source, wherein the collagen source includes proteins or peptides including at least 5 percent proline residues, at least 5 percent hydroxyproline residues, and at least 20 percent glycine residues based on a total weight average of the proteins or peptides;
   (b) a glutamate source;
   (c) a cysteine source;
   (d) a selenium source; and
   (e) a boron source.
      In some embodiments, the step of administering includes administering to the subject a dose of the glutathione support composition, wherein the dose is about 1 gram to about 5 grams of the glutathione support composition. In other embodiments, the step of administering includes administering the dose to the subject daily for a dosing period of at least 3 consecutive days. In yet other embodiments, the subject has a ratio of intracellular reduced glutathione (GSH) to oxidized glutathione (GSSH) of at least 5:1 after the dosing period. In still other embodiments, the dose includes about 800 to about 3,000 mg of the collagen source, about 400 to about 2,000 mg of the glutamate source, and about 200 to about 1,000 mg of the cysteine source. In some aspects, the collagen source is selected from the group consisting of collagen, collagen peptides, gelatin, gelatin peptides, elastin, elastin peptides, glycine-rich RNA-binding proteins, tenascin-C, proteoglycans, hydrolysates thereof (peptides thereof), and combinations thereof. In other aspects, the glutamate source includes L-glutamine, a L-glutamine precursor, glutamic acid, a glutamic acid precursor, a salt thereof, or a combination thereof. In yet other aspects, the cysteine source includes L-cystine, N-acetyl cysteine, a salt thereof, or a combination thereof. In other embodiments, the selenium source includes selenomethionine, and the dose includes the selenomethionine in a catalytically effective amount. In still other embodiments, the glutathione support composition further includes one or more additional active ingredients selected from the group consisting of a zinc source, methylfolate, methyl-B₁₂, quercetin, sulforaphane, methionine, vitamin D, vitamin k2, vitamin C, L-threonine, L-theanine, turmeric, curcumin, taurine, melatonin, ashwagandha, fisetin, 1-NMA, NAD+, or kaempferol (or other flavonoid antioxidants), or a combination thereof. In yet other embodiments, the glutathione support composition further includes taurine, melatonin, sulforaphane, quercetin, Vitamin D3, Vitamin k2, Vitamin C, metformin, L-threonine, L-theanine, turmeric, curcumin, ashwagandha or an extract thereof, fish oil, krill oil, omega 3 fatty acids, odd-chain fatty acids, botanical extracts, or a combination thereof.

The present disclosure also relates to a method for reducing gamma-glutamyltransferase (GGT) levels in a subject, including:
administering to the subject a dosing regimen of a glutathione support composition including:
   (a) about 30 to about 65 weight percent of a collagen source (based on the total weight of the glutathione support composition), wherein the collagen source includes proteins or peptides including at least 5 percent proline residues, at least percent hydroxyproline residues, and at least 20 percent glycine residues based on a total weight average of the proteins or peptides;
   (b) about 25 to about 50 weight percent of a glutamate source (based on the total weight of the glutathione support composition);
   (c) about 10 to about 30 weight percent of a cysteine source (based on the total weight of the glutathione support composition);
   (d) a selenium source; and
   (e) a boron source.

In some embodiments, the dosing regimen includes administering a dose of a therapeutically effective amount of the glutathione support composition for a dosing period of at least three consecutive days. In other embodiments, the subject has a ratio of intracellular reduced glutathione (GSH) to oxidized glutathione (GSSH) of at least 5:1 after the dosing period. In still other embodiments, the collagen source includes collagen, collagen peptides, gelatin, gelatin peptides, elastin, elastin peptides, glycine-rich RNA-binding proteins, tenascin-C, proteoglycans, hydrolysates thereof (peptides thereof), or combinations thereof. In yet other embodiments, the collagen source includes collagen peptides, gelatin peptides, elastin peptides, or a combination thereof. In some aspects, the collagen source includes hydrolyzed marine or ovine collagen, hydrolyzed marine or ovine gelatin, hydrolyzed elastin, or a mixture thereof.

The present disclosure further relates to a method for reducing gamma-glutamyltransferase (GGT) levels in a subject, including:
administering to the subject a dose of a glutathione support composition including:
   (a) about 30 to about 65 weight percent of a collagen source (based on the total weight of the glutathione support composition), wherein the collagen source includes hydrolyzed marine or ovine collagen, hydrolyzed marine or ovine gelatin, hydrolyzed elastin, or a mixture thereof
   (b) about 25 to about 50 weight percent of a glutamate source (based on the total weight of the glutathione support composition);
   (c) about 10 to about 30 weight percent L-cystine (based on the total weight of the glutathione support composition);
   (d) a selenium source; and
   (e) a boron source.

In some embodiments, the glutamate source includes L-glutamine, a L-glutamine precursor, glutamic acid, a glutamic acid precursor, a salt thereof, or a combination thereof. In other embodiments, the step of administering includes administering the dose to the subject daily for a dosing period of at least 3 consecutive days, and wherein the subject has a ratio of intracellular reduced glutathione (GSH) to oxidized glutathione (GSSH) of at least 5:1 after the dosing period.

The present disclosure also relates to a method for treating tuberculosis or MRSA in a subject, including:
administering to the subject a therapeutically effective dose of a glutathione support composition including:
   (a) a collagen source, wherein the collagen source includes proteins or peptides including at least 5 percent proline residues, at least 5 percent hydroxyproline residues, and at least 20 percent glycine residues based on a total weight average of the proteins or peptides;
   (b) a glutamate source;
   (c) a cysteine source;
   (d) a selenium source; and
   (e) a boron source.

In some embodiments, the step of administering includes administering the dose to the subject daily for a dosing period of at least 5 consecutive days. In other embodiments, the step of administering occurs concurrently with treating the subject with an antibiotic agent. In yet other embodiments, the antibiotic agent includes isoniazid, rifampin, amikacin, aminosalicylic acid, capreomycin, cycloserine, ethambutol, ethionamide, kanamycin, pyrazinamide, rifapentine, rifabutin, streptomycin, ofloxacin, ciprofloxacin, clarithromycin, azithromycin, fluoroquinolones, or a combination thereof. In still other embodiments, the antibiotic agent includes cephapirin, amoxicillin, trimethoprim-sulfonamides, sulfonamides, oxytetracycline, fluoroquinolones, enrofloxacin, danofloxacin, marbofloxacin, cefquinome, ceftiofur, streptomycin, oxytetracycline, vancomycin, cefazolin, cephalothin, cephalexin, linezolid, daptomycin, clindamycin, lincomycin, mupirocin, bacitracin, neomycin, polymyxin B, gentamicin, prulifloxacin, ulifloxacin, fidaxomicin, minocycline, metronidazole, metronidazole, sulfamethoxazole, ampicillin, trimethoprim, ofloxacin, norfloxacin, tinidazole, norfloxacin, ornidazole, levofloxacin, nalidixic acid, ceftriaxone, azithromycin, cefixime, ceftriaxone, cefalexin, ceftriaxone, rifaximin, ciprofloxacin, norfloxacin, ofloxacin, levofloxacin, gatifloxacin, gemifloxacin, prufloxacin, ulifloxacin, moxifloxacin, nystatin, amphotericin B, flucytosine, ketoconazole, posaconazole, clotrimazole, voriconazole, griseofulvin, miconazole nitrate, fluconazole, or a combination thereof. In yet other embodiments, the antibiotic agent includes an oxazolidinone compound. In some aspects, the dose includes about 800 to about 3,000 mg of the collagen source, about 400 to about 2,000 mg of the glutamate source, and about 200 to about 1,000 mg of the cysteine source. In other aspects, the collagen source is selected from the group consisting of collagen, collagen peptides, gelatin, gelatin peptides, elastin, elastin peptides, glycine-rich RNA-binding proteins, tenascin-C, proteoglycans, hydrolysates thereof (peptides thereof), and combinations thereof. In yet other aspects, the cysteine source includes L-cystine, N-acetyl cysteine, a salt thereof, or a combination thereof.

The present disclosure further relates to a method for preventing infection with tuberculosis or MRSA in a subject, including:
administering to the subject a therapeutically effective dose of a glutathione support composition including:
   (a) about 30 to about 65 weight percent of a collagen source (based on the total weight of the glutathione support composition), wherein the collagen source includes proteins or peptides including at least 5 percent proline residues, at least percent hydroxyproline residues, and at least 20 percent glycine residues based on a total weight average of the proteins or peptides;
   (b) about 25 to about 50 weight percent of a glutamate source (based on the total weight of the glutathione support composition);
   (c) about 10 to about 30 weight percent of a cysteine source (based on the total weight of the glutathione support composition);
   (d) a selenium source; and
   (e) a boron source.
      In some embodiments, the step of administering includes administering the dose to the subject daily for a dosing period of at least three consecutive days. In other embodiments, the collagen source includes collagen, collagen peptides, gelatin, gelatin peptides, elastin, elastin peptides, glycine-rich RNA-binding proteins, tenascin-C, proteoglycans, hydrolysates thereof (peptides thereof), or combinations thereof. For example, the collagen source may include collagen peptides, gelatin peptides, elastin peptides, or a combination thereof. In some aspects, the collagen source includes hydrolyzed marine or ovine collagen, hydrolyzed marine or ovine gelatin, hydrolyzed elastin, or a mixture thereof. In other aspects, the glutamate source includes L-glutamine, a L-glutamine precursor, glutamic acid, a glutamic acid precursor, a salt thereof, or a combination thereof. In yet other aspects, the cysteine source includes L-cystine, N-acetyl cysteine, a salt thereof, or a combination thereof.

The present disclosure also relates to a method for preventing or treating infection with tuberculosis or MRSA in a subject, including:
administering to the subject a dose of a glutathione support composition including:
   (a) about 30 to about 65 weight percent of a collagen source (based on the total weight of the glutathione support composition), wherein the collagen source includes hydrolyzed marine or ovine collagen, hydrolyzed marine or ovine gelatin, hydrolyzed elastin, or a mixture thereof;
   (b) about 25 to about 50 weight percent of a glutamate source (based on the total weight of the glutathione support composition);
   (c) about 10 to about 30 weight percent of a cysteine source (based on the total weight of the glutathione support composition);
   (d) a selenium source; and
   (e) a boron source.
      In some embodiments, the dose includes about 800 to about 3,000 mg of the collagen source, about 400 to about 2,000 mg of the glutamate source, and about 200 to about 1,000 mg of the cysteine source. In other embodiments, the step of administering includes administering the dose to the subject daily for a dosing period of at least 5 consecutive days. In yet other embodiments, the glutamate source includes L-glutamine, a L-glutamine precursor, glutamic acid, a glutamic acid precursor, a salt thereof, or a combination thereof.

The present disclosure also relates to a method for improving vaccine therapy in a subject, including:
administering to the subject a therapeutically effective dose of a glutathione support composition including:
   (a) a collagen source, wherein the collagen source includes proteins or peptides including at least 5 percent proline residues, at least 5 percent hydroxyproline residues, and at least 20 percent glycine residues based on a total weight average of the proteins or peptides;
   (b) a glutamate source;
   (c) a cysteine source;
   (d) a selenium source; and
   (e) a boron source.
      In some embodiments, the step of administering includes administering to the subject a daily dose of the glutathione support composition at least five consecutive days prior to the subject receiving a dose of a vaccine. In other embodiments, the step of administering includes administering to the subject a daily dose of the glutathione support composition at least five consecutive days prior to the subject receiving a dose of a vaccine and five consecutive days after the subject receives a dose of a vaccine. In yet other embodiments, the step of administering includes administering to the subject a daily dose of the glutathione support composition at least five consecutive days after the subject receives a dose of a vaccine. In some aspects, the dose includes about 800 to about 3,000 mg of the collagen source, about 400 to about 2,000 mg of the glutamate source, and about 200 to about 1,000 mg of the cysteine source. In other aspects, the collagen source is selected from the group consisting of collagen, collagen peptides, gelatin, gelatin peptides, elastin, elastin peptides, glycine-rich RNA-binding proteins, tenascin-C, proteoglycans, hydrolysates thereof (peptides thereof), and combinations thereof. In yet other aspects, the glutamate source includes L-glutamine, a L-glutamine precursor, glutamic acid, a glutamic acid precursor, a salt thereof, or a combination thereof. In still other aspects, the cysteine source includes L-cystine, N-acetyl cysteine, a salt thereof, or a combination thereof. In other embodiments, the selenium source includes selenomethionine, and the dose includes the selenomethionine in a catalytically effective amount. In yet other embodiments, the glutathione support composition further includes taurine, melatonin, sulforaphane, quercetin, Vitamin D3, Vitamin k2, Vitamin C, 1-NMA, metformin, L-threonine, L-theanine, turmeric, curcumin, ashwagandha or an extract thereof, fish oil, krill oil, omega 3 fatty acids, odd-chain fatty acids, botanical extracts, or a combination thereof.

The present disclosure further relates to a method for improving vaccine therapy in a subject, including:
administering to the subject a therapeutically effective dose of a glutathione support composition including:
   (a) about 30 to about 65 weight percent of a collagen source (based on the total weight of the glutathione support composition), wherein the collagen source includes proteins or peptides including at least 5 percent proline residues, at least percent hydroxyproline residues, and at least 20 percent glycine residues based on a total weight average of the proteins or peptides;
   (b) about 25 to about 50 weight percent of a glutamate source (based on the total weight of the glutathione support composition);
   (c) about 10 to about 30 weight percent of a cysteine source (based on the total weight of the glutathione support composition);
   (d) a selenium source; and
   (e) a boron source.
      In some embodiments, the step of administering includes administering the dose to the subject daily for a dosing period that begins before the subject receives a dose of one or more vaccines and ends after the subject receives the dose of the one or more vaccines. In other embodiments, the dosing period is at least 10 days. In still other embodiments, the dosing period is at least 6 days. In yet other embodiments, the collagen source includes collagen, collagen peptides, gelatin, gelatin peptides, elastin, elastin peptides, glycine-rich RNA-binding proteins, tenascin-C, proteoglycans, hydrolysates thereof (peptides thereof), or combinations thereof. In some aspects, the collagen source includes collagen peptides, gelatin peptides, elastin peptides, or a combination thereof. In other aspects, the collagen source includes hydrolyzed marine or ovine collagen, hydrolyzed marine or ovine gelatin, hydrolyzed elastin, or a mixture thereof.

The present disclosure also relates to a method for improving response to vaccine therapy in a subject, including:
administering to the subject a pre-vaccine dose of a glutathione support composition including:
(a) a collagen source;
(b) a glutamate source;
(c) a cysteine source;
(d) a selenium source; and
(e) a boron source,
administering to the subject a vaccine dose of the glutathione support composition; and
administering to the subject a post-vaccine dose of the glutathione support composition, wherein the pre-vaccine dose is administered daily for a period of at least 3 days prior to the subject receiving one or more vaccines, wherein the vaccine dose occurs concurrently with the subject receiving the one or more vaccines, and wherein the post-vaccine dose is administered daily for a period of at least 3 days after the subject has received the one or more vaccines.
   In some embodiments, the collagen source includes collagen, collagen peptides, gelatin, gelatin peptides, elastin, elastin peptides, glycine-rich RNA-binding proteins, tenascin-C, proteoglycans, hydrolysates thereof (peptides thereof), or combinations thereof, wherein the glutamate source L-glutamine, a L-glutamine precursor, glutamic acid, a glutamic acid precursor, a salt thereof, or a combination thereof, and wherein the cysteine source includes L-cystine, N-acetyl cysteine, a salt thereof, or a combination thereof. In other embodiments, the collagen source includes hydrolyzed marine or ovine collagen, hydrolyzed marine or ovine gelatin, hydrolyzed elastin, or a mixture thereof.

### BRIEF DESCRIPTION OF THE DRAWINGS

The following drawings are attached to—and form a portion of—this disclosure. Implementation of the compositions and methods of use thereof are described, by way of example only, with reference to the following figures:

FIGS. 1A-4B show a reduction in gamma-glutamyltransferase (GGT) levels and high-sensitivity C-reactive protein (hs-CRP) after administration of the glutathione support compositions made in accordance with an embodiment of the present disclosure.

It should be understood that the various aspects are not limited to the compositions or results shown in the drawings.

### DETAILED DESCRIPTION OF THE INVENTION

Glutathione (L-γ-glutamyl-L-cysteinylglycine) (GSH or glutathione), shown in Formula I below

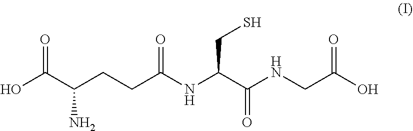 [SMILES: N[C@@H](CCC(=O)N[C@@H](CS)C(=O)NCC(=O)O)C(=O)O]

is essential for proper functioning of the immune system and is vital for building and repairing tissue. It acts as an important antioxidant, which helps protect the body from damage to cells caused by free radicals. More specifically, glutathione plays a central role in shielding cellular macromolecules from endogenous and exogenous reactive oxygen and nitrogen species. The glutathione support compositions of the present disclosure maintain or enhance intracellular glutathione, which allows the body to naturally fend off cellular damage, among other benefits.

In the context of the present disclosure, a “glutathione support composition” refers to a composition that is administered to an individual and increases intracellular glutathione levels in the individual beyond existing levels. The components of the glutathione support compositions, methods of administration, and methods of use are described in more detail below.

### The Formulation

The glutathione support compositions include a collagen source, a glutamate source, a cysteine source, a selenium source, and, optionally, boron.

Collagen Source

As generally discussed above, the compositions of the present disclosure include a collagen source. The collagen source is preferably selected from proteins or peptides that include proline residues, hydroxyproline residues, and glycine residues, but the collagen source may also be another glycine precursor such as dimethylglycine, serine, thereonine, combinations thereof, or combinations with the proteins or peptides discussed in this section. Without wishing to be bound to any particular theory, it is believed that the proteins or peptides are digested and subsequently absorbed into by the body in a more effective and sustained manner, such that ample glycine is continuously available for glutathione synthesis. In addition to contributing to an increase in glutathione synthesis, the proteins and peptides provide additional health benefits. For example, the proteins or peptides improve health and slow the effects of aging by stimulating collagen production, which requires high amounts of proline, hydroxyproline, and glycine. Increased collagen synthesis contributes to a reduction of wrinkles, rejuvenation of skin, and reversal of skin aging by improving skin hydration and elasticity. Protein and peptides containing hydroxyproline and glycine have been shown to repair muscle damage, improve sleep quality, improve joint health, and improve would healing.

In some embodiments, the proteins or peptides include at least about 5 weight percent proline residues, at least about 5 weight percent hydroxyproline residues, and at least about 20 weight percent glycine residues (based on a total weight average of the proteins or peptides). The proteins or peptides can optionally be glycosylated.

In some aspects, the proteins or peptides include at least about 5 weight percent proline residues (based on a total weight average of the proteins or peptides). For example, the proteins or peptides may include at least about 7 weight percent, about 8 weight percent, at least about 9 weight percent, or at least about 10 weight percent of proline residues (based on a total weight average of the proteins or peptides). In other aspects, the proteins or peptides include at least about 5 weight percent hydroxyproline residues (based on a total weight average of the proteins or peptides). For example, the proteins or peptides may include at least about 6 weight percent, about 8 weight percent, at least about 10 weight percent, at least about 12 weight percent, or at least about 14 weight percent of hydroxyproline residues (based on a total weight average of the proteins or peptides). In still other aspects, the proteins or peptides include at least about 20 weight percent glycine residues (based on a total weight average of the proteins or peptides). For example, the proteins or peptides may include at least about 23 weight percent, about 24 weight percent, at least about 25 weight percent, or at least about 28 weight percent of glycine residues (based on a total weight average of the proteins or peptides).

In some embodiments, the proteins or peptides comprise:
at least about 5 weight percent proline residues, at least about 5 weight percent hydroxyproline residues, and at least about 23 weight percent glycine residues;
at least about 6 weight percent proline residues, at least about 8 weight percent hydroxyproline residues, and at least about 25 weight percent glycine residues;
at least 8 weight percent proline residues, at least 8 weight percent hydroxyproline residues, and at least 25 weight percent glycine residues;
at least about 8 weight percent proline residues, at least about 10 weight percent hydroxyproline residues, and at least about 25 weight percent glycine residues;
at least about 5 weight percent proline residues, at least about 5 weight percent hydroxyproline residues, and at least about 30 weight percent glycine residues;
at least about 6 weight percent proline residues, at least about 8 weight percent hydroxyproline residues, and at least about 30 weight percent glycine residues;
at least about 8 weight percent proline residues, at least about 8 weight percent hydroxyproline residues, and at least about 30 weight percent glycine residues;
at least about 8 weight percent proline residues, at least about 10 weight percent hydroxyproline residues, and at least about 30 weight percent glycine residues; or
at least about 8 weight percent proline residues, at least about 12 weight percent hydroxyproline residues, and at least about 30 weight percent glycine residues, based on a total weight average of the proteins or peptides.

Nonlimiting examples of proteins or peptides comprising at least about 5 weight percent proline residues, at least about 5 weight percent hydroxyproline residues, and at least about 20 weight percent glycine residues include collagen, gelatin, elastin, glycine-rich RNA-binding proteins, tenascin-C, proteoglycans, hydrolysates thereof (peptides thereof), or a combination thereof. In various embodiments, the proteins or peptides are selected from collagen, hydrolyzed collagen, gelatin, hydrolyzed gelatin, elastin, hydrolyzed elastin, or a mixture thereof. In some embodiments, the one or more proteins or peptides are selected from hydrolyzed collagen, hydrolyzed gelatin, hydrolyzed elastin, or a mixture thereof.

The term “hydrolyzed collagen” is interchangeable with the terms “collagen hydrolysate” and “collagen peptides.” The term “hydrolyzed gelatin” is interchangeable with the term “gelatin hydrolysate” and “gelatin peptides.” The term “hydrolyzed elastin” is interchangeable with the term “elastin hydrolysate” and “elastin peptides.”

Collagen

In recent years, collagen has been found to exert various physiological and pharmacological effects such as bone strengthening effects that lead to prevention and/or improvement of osteoporosis, effects for accelerating metabolism in living tissue, which ameliorate the declining functions of living tissues with aging, skin metabolism accelerating effects, skin activating effects, and antiaging effects for skin in order to prevent and/or improve wrinkles. In the context of the present disclosure, collagen may be used as the collagen source. Any of the 30 or more types of collagen may be used in accordance with the invention. In some embodiments, the collagen source is Type I, Type II, or Type IV collagen.

Collagen Peptides/Hydrolyzed Collagen

The collagen source may also be hydrolyzed collagen. In this aspect, the hydrolyzed collagen can be Type I, Type II, Type III, Type IV, or Type V. In various embodiments, the hydrolyzed collagen is preferably Type I, Type III, or a combination thereof. Furthermore, collagen can be derived from a variety of sources. For example, it may be derived from beef, chicken, fish, eggshell membrane, the skin or other connective tissue of sheep, and chicken egg whites. In various embodiments, the source of the collagen is fish, referred to as marine collagen. In this aspect, marine collagen peptides Type I are particularly useful. In other embodiments, the source of the collagen is sheep, referred to as ovine collagen.

The average molecular weight of the collagen peptides can vary. Nonetheless, in various embodiments, the average molecular weight is 500 to 20,000 Daltons. In various embodiments, the average molecular weight is about 500 to about 15,000 Dalton, about 500 to about 12,500 Dalton, about 500 to about 11,000 Daltons, about 500 to about 10,000 Daltons, about 500 to about 8,000 Dalton, about 500 to about 5,000 Daltons. In various embodiments, it is preferable if the average molecular weight of the collagen peptides is not more than 10,000 Dalton, not more than 8,000 Daltons, not more than 5,000 Daltons, not more than 2,000 Dalton, or not more than 1,000 Daltons.

Gelatin

Gelatin is also a suitable collagen source in accordance with the present disclosure. It may be obtained through the controlled and partial hydrolysis of collagen from animal skin, bone, and tissue. The composition of gelatin is determined by the amino acid sequence of its collagen source, e.g., when derived from Type I collagen, the gelatin consists of a sequence of amino acids in the form of a triple helix.

Gelatin Peptides/Hydrolyzed Gelatin

Gelatin peptides are also suitable for use as the collagen source. In fact, the functional properties of gelatin can be improved by enzymatic hydrolysis for conversion to peptides. The amino acid composition of the gelatin peptides is very similar to that of the parent proteins, which are rich in glycine, alanine, proline, aspartic acid, and glutamic acid. High glycine and proline contents make the antioxidant activity of fish skin gelatin higher than that of meat protein. Accordingly, in various embodiments gelatin peptides derived from marine collagen are particularly useful. In fact, marine collagen peptides and marine gelatin peptides have been associated with accelerated wound healing in addition to anti-aging properties. For example, in humans, marine collagen peptides and marine gelatin peptides contribute to reduce wrinkles, improve skin elasticity, and enhance the overall structure and appearance of skin. As such in some embodiments, the collagen source is a combination of marine collagen peptides and marine gelatin peptides.

The average molecular weight of the gelatin peptides can vary. Nonetheless, in various embodiments, the average molecular weight is 500 to 20,000 Daltons. In various embodiments, the average molecular weight is about 500 to about 15,000 Daltons, about 500 to about 12,500 Daltons, about 500 to about 11,000 Daltons, about 500 to about 10,000 Daltons, about 500 to about 8,000 Daltons, about 500 to about 5,000 Daltons. In various embodiments, it is preferable if the average molecular weight of the gelatin peptides is not more than 10,000 Daltons, not more than 8,000 Daltons, not more than 5,000 Daltons, not more than 2,000 Daltons, or not more than 1,000 Daltons.

Elastin and Elastin Peptides/Hydrolyzed Elastin

Elastin is one of the most abundant proteins in the human body. The main amino acids of elastin include proline, glycine, desmosine, and isodesmosine. In some embodiments, elastin is used as the collagen source.

Hydrolysis of the elastin results in peptides with an average molecular weight about 1,000 to about 5,000 Daltons. This makes it easily soluble and easily absorbed in the body. Significant amounts of elastin peptides are seen in the skin even 48 hours after oral intake. Elastin peptides are also powerful antioxidants. In some embodiments, the collagen source includes elastin peptides.

The average molecular weight of the elastin peptides can vary. Nonetheless, in various embodiments, the average molecular weight is 500 to 20,000 Daltons. In various embodiments, the average molecular weight is about 500 to about 15,000 Dalton, about 500 to about 12,500 Dalton, about 500 to about 11,000 Daltons, about 500 to about 10,000 Daltons, about 500 to about 8,000 Dalton, about 500 to about 5,000 Daltons. In various embodiments, it is preferable if the average molecular weight of the elastin peptides is not more than 10,000 Dalton, not more than 8,000 Daltons, not more than 5,000 Daltons, not more than 2,000 Dalton, or not more than 1,000 Daltons.

The total amount of the collagen source in the glutathione support compositions will vary. Nonetheless, in various embodiments, the collagen source is present in an amount higher than any other single component of the glutathione support compositions. For example, in various embodiments, the glutathione support compositions include at least about 25 weight percent of the collagen source (based on the total weight of the glutathione support composition). In further embodiments, the glutathione support composition includes about 25 to about 85 weight percent, about 25 to about 80 weight percent, about 25 to about 70 weight percent, about 25 to about 60 weight percent, about 25 to about 50 weight percent, about 25 to about 40 weight percent, about 30 to about 85 weight percent, about 30 to about 70 weight percent, about 30 to about 60 weight percent, about 30 to about 50 weight percent, about 35 to about 85 weight percent, about 35 to about 80 weight percent, about 35 to about 70 weight percent, about 35 to about 60 weight percent, about 40 to about 85 weight percent, about 40 to about 80 weight percent, about 40 to about 70 weight percent, about 40 to about 60 weight percent, about 40 to about 55 weight percent of the collagen source (based on the total weight of the glutathione support composition).

The daily dosage amount of the collagen source will vary depending on a variety of factors. Nonetheless, in various embodiments, the daily dosage amount of the collagen source may be from about 100 mg to about 4,000 mg. In further embodiments, the daily dosage amount is from about 200 to about 4,000 mg, about 500 to about 4,000 mg, about 1,000 to about 4,000 mg, about 100 to about 3,000 mg, about 200 to about 3,000 mg, about 500 to about 3,000 mg, about 1,000 to about 3,000 mg, about 100 to about 2,000 mg, about 200 to about 2,000 mg, about 500 to about 2,000 mg, about 1,000 to about 2,000 mg, about 100 to about 1,500 mg, about 200 to about 1,500 mg, about 500 to about 1,500 gm, or about 1,000 to about 1,500 mg.

Glutamate Source

L-glutamine is the most abundant and versatile amino acid in the body, and is of fundamental importance to intermediary metabolism, interorgan nitrogen exchange via ammonia (NH₃) transport between tissues, and pH homeostasis. In almost every cell, glutamine can be used as a substrate for nucleotide synthesis (purines, pyrimidines, and amino sugars), nicotinamide adenine dinucleotide phosphate (NADPH), antioxidants, and many other biosynthetic pathways involved in the maintenance of cellular integrity and function.

L-glutamine is readily absorbed by the body and may be useful as the glutamate source in the glutathione support compositions of the present disclosure. Nonetheless, L-glutamine precursors can also be readily absorbed by the body and in some instance, provide better intracellular sources of L-glutamine than administration of L-glutamineper se. Nonlimiting examples of L-glutamine precursors that may be used as the glutamate source include esters of L-glutamine, glutamic acid, esters of glutamic acid, glutamine dipeptides (for example glutamine-glutamine dipeptide, glycyl-L-glutamine dipeptide, etc.), N-acetylglutamine (NAG), ornithine, alpha-ketoglutarate (AKG), a salt thereof, or a combination thereof. As used herein, the term “a salt thereof” is interchangeable with the term “salts thereof” Thus, where the disclosure refers to “an element selected from A, B, C, D, E, F, a salt thereof, or a combination thereof,” it indicates that that one or more of A, B, C, D, and F may be included, one or more of a salt of A, a salt of B, a salt of C, a salt of D, a salt of E, and a salt of F may be included, or a combination of any two of A, B, C, D, E, F, a salt of A, a salt of B, a salt of C, a salt of D, a salt of E, and a salt of F may be included.

Nonlimiting examples of salts of glutamic acid include monosodium glutamate and monopotassium glutamate. Nonlimiting examples of esters of L-glutamine include L-glutamine methyl ester and L-glutamine methyl ester hydrochloride, L-glutamine t-butyl ester, and L-glutamine t-butyl ester hydrochloride. Nonlimiting examples of esters of glutamic acid include L-glutamic acid 1-methyl esters, L-glutamic acid dimethyl ester, and L-glutamic acid 5-ethyl ester. Nonlimiting examples of L-glutamine dipeptides include L-glycyl-L-glutamine (Gly-Gln), L-arginyl-L-glutamine (Arg-Gln) and L-alanyl-L-glutamine (Ala-Gln). L-glycyl-L-glutamine (Gly-Gln) is particularly useful because it provides an L-glycine residue in addition to a L-glutamine residue.

The total amount of the glutamate source in the glutathione support composition will vary. Nonetheless, in various embodiments, the glutathione support composition includes about 5 to about 50 weight percent of the glutamate source based on the total weight of the glutathione support composition. In further embodiments, the glutathione support composition includes about 5 to about 45 weight percent, about 5 to about 40 weight percent, about 5 to about 35 weight percent, about 10 to about 50 weight percent, about 10 to about 45 weight percent, about 10 to about 40 weight percent, about 10 to about 35 weight percent, about 15 to about 50 weight percent, about 15 to about 45 weight percent, about 15 to about 40 weight percent, about 15 to about 35 weight percent, about 20 to about 50 weight percent, about 20 to about 45 weight percent, about 20 to about 40 weight percent, about 20 to about 35 weight percent, about 25 to about 50 weight percent, about 25 to about 45 weight percent, about 25 to about 40 weight percent, or about 25 to about 35 weight percent, of the glutamate source (based on the total weight of the glutathione support composition).

The daily dosage amount of the glutamate source will vary depending on a variety of factors. Nonetheless, in various embodiments, the daily dosage amount of the glutamate source may be from about 100 mg to about 2,000 mg. In further embodiments, the daily dosage amount is from about 200 to about 2,000 mg, about 500 to about 2,000 mg, about 100 to about 1,500 mg, about 200 to about 1,500 mg, about 500 to about 1,500 mg, about 100 to about 1,200, about 200 to about 1,200 mg, about 500 to about 1,200 mg, about 100 to about 1,000 mg, about 200 to about 1,000 mg, about 500 to about 1,000 mg, or about 600 to about 1,200 mg.

Cysteine Source

The bioavailability of L-cysteine, one of the three amino acids used to form glutathione, is low because it readily undergoes oxidation in the digestive tract. Thus, the cysteine source used in the formulation of the present disclosure preferably has a high bioavailability than L-cysteine. In this aspect, the cysteine source may be L-cystine, another L-cysteine precursor, a salt thereof, or a combination thereof.

L-cystine is particularly useful because it provides two L-cysteine residues. L-cystine includes a disulfide bond between two cysteine residues, which is reduced in the body into two L-cysteines that are immediately available for glutathione synthesis. L-cystine also has a higher extracellular concentration and hence it is favored with the diffusion gradient into the cell. This allows the L-cystine to get into the cell where it can then provide two L-cysteine amino acids to the cell for glutathione production. L-cysteine, on the other hand, has a higher intracellular concentration and is therefore less likely to easily diffuse into cells. The diffusion gradient for L-cysteine from extracellular to intracellular concentration is low due to several properties. L-cysteine is a relatively large and polar amino acid that cannot easily pass through the hydrophobic lipid bilayer. This causes L-cysteine to be reliant on specific transporters to cross the membrane. Additionally, L-cysteine is subject to oxidation and degradation in the extracellular space, reducing its availability for uptake into the cells.

Nonlimiting examples of other L-cysteine precursors include N-acetyl cysteine, L-cysteine esters, for example, L-cysteine methyl ester or L-cysteine ethyl ester, methionine, salts thereof, anhydrides thereof, and combinations thereof. Other nonlimiting examples of L-cysteine precursors or sulfur containing compounds that may be used with or instead of an L-cysteine precursor include allicin, dially disulfide (DDS), S-allylcysteine, S-methylcysteine (SMC), a mixtures thereof.

The total amount of the cysteine source in the glutathione support composition will vary. Nonetheless, in various embodiments, the total amount of the L-cystine, other L-cysteine precursor (and/or sulfur containing compounds), salt thereof, or a combination thereof is about 5 to about 50 weight percent (based on the total weight of the glutathione support composition). In further embodiments, the glutathione support composition includes about 5 to about 40 weight percent, about 5 to about 35 weight percent, about 5 to about 30 weight percent, about 5 to about weight percent, about 5 to about 20 weight percent, about 10 to about 50 weight percent, about 10 to about 40 weight percent, about 10 to about 35 weight percent, about 10 to about 30 weight percent, about 10 to about 25 weight percent, about 10 to about 20 weight percent, about 12 to about 50 weight percent, about 12 to about 40 weight percent, about 12 to about 35 weight percent, about 12 to about 30 weight percent, about 12 to about 25 weight percent, about 12 to about 20 weight percent, or about 12 to about 18 weight percent of the cysteine source (based on the total weight of the glutathione support composition).

The daily dosage amount of the cysteine source will vary depending on a variety of factors. Nonetheless, in various embodiments, the daily dosage amount of the cysteine source may be from about 100 mg to about 2,000 mg. In further embodiments, the daily dosage amount is from about 200 to about 2,000 mg, about 500 to about 2,000 mg, about 100 to about 1,500 mg, about 200 to about 1,500 mg, about 500 to about 1,500 mg, about 100 to about 1,200, about 200 to about 1,200 mg, about 500 to about 1,200 mg, about 100 to about 1,000 mg, about 200 to about 1,000 mg, about 100 to about 800 mg, or about 100 to about 500 mg.

Selenium Source

The addition of a selenium source to the compositions is also beneficial, as selenium serves as a mineral cofactor in glutathione biosynthesis. The term “selenium source” in the context of the instant disclosure is a physiologically acceptable material that delivers selenium to the body, preferably when orally consumed. The selenium source can be a compound, a salt, a chelate, or a composition. Nonlimiting examples selenium sources include selenium, selenomethionine, L-selenomethionine, selenocysteine, L-selenocysteine, methyl selenocysteine, selenium nanoparticles, selenite, Se-methyl selenocysteine, methylselenol, amino acid chelates of selenium, dimethyl selenium, trimethyl selenium, seleno-methyl selenocysteine, selenocystathionine, selenotaurine, selenodiglutathione, allyl selenocysteine, propyl selenocysteine, selenoethionine and selenocystamine, yeast (selenium yeast), sodium selenate, sodium selenite, selenocyanate, L-selenomethionine, Se-methylseleno-L-cysteine, L-selenohomolanthionine, L-selenocystine, Se-methylseleno-N-acetylgalactosamine, trimethylselenonium ion, kelp bound selenium, and combinations thereof.

In some embodiments, the selenium source is selected from selenomethionine, in particular L-selenomethionine, L-selenocystine, L-selenocysteine, Se-methylseleno-L-cysteine, or a combination thereof.

In other embodiments, the selenium source is a synthetic organoselenium compound. For example, a synthetic organoselenium compound suitable for use as the selenium source is ebselen. In still other embodiments, the selenium source is a selenoprotein such as selenoprotein P or thioredoxin reductase. In yet other embodiments, the selenium-containing component may an inorganic selenium compound such as an aliphatic metal salt containing selenium in the form of selenite or selenate anions.

The amount of the selenium source in the glutathione support composition will vary, for example, depending on the selenium source. Some selenium sources include higher amounts of selenium than other selenium sources. Nonetheless, in various embodiments, the glutathione support composition includes a selenium source in an amount such that the glutathione support composition includes about 1 to about 300 ppm of selenium. In further embodiments, the glutathione support composition includes a selenium source in an amount such that the glutathione support composition includes about 1 to about 200 ppm, about 5 to about 300 ppm, about 5 to about 200 ppm, about 10 to about 300 ppm, about 10 to about 200 ppm, about 25 to about 300 ppm, about 25 to about 200 ppm, about 30 to about 300 ppm, about 30 to about 200 ppm, about 30 to about 100 ppm, or about 30 to about 50 ppm of elemental selenium. The daily dosage amount of the selenium source will vary depending on a variety of factors. Nonetheless, in various embodiments, the daily dosage amount of the selenium source provides about 1 to about 100 μg of elemental selenium. In further embodiments, the daily dosage amount of the selenium source provides 1 to about 80 μg, about 1 to about 60 μg, about 1 to about 50 μg, about 1 to about 25 μg, about to about 20 μg, about 2 to about 100 μg, about 2 to about 80 μg, about 2 to about 60 μg, about 2 to about 50 μg, about 2 to about 25 μg, about 2 to about 25 μg, about 2 to about 20 μg, about 5 to about 100 μg, about 5 to about 80 μg, about 5 to about 60 μg, about 5 to about 50 μg, about 5 to about 25 μg, or about 5 to about 20 μg.

Boron Source

The glutathione support compositions may also include a boron source. While boron alone is not known to appreciably impact or influence glutathione synthesis, the addition of boron source to the glutathione support compositions surprisingly enhances its glutathione potentiating ability, in addition to providing several other beneficial effects. For example, the source of boron contributes to increasing the expression of certain enzymes involved in glutathione metabolism, such as gamma-glutamyl transferase.

Without being bound by any particular theory, boron is believed to have a synergistic-like effect when included in the glutathione support compositions of the instant disclosure through a multiple of possible pathways. One mechanism by which boron may increase cysteine uptake and utilization of cysteine by cells for glutathione intracellular production is by increasing the expression of cysteine transporters, which are proteins that facilitate the transport of cysteine into the cell. Studies have shown that boron can increase the expression of the cysteine transporter system, which is responsible for the transport of cysteine into cells in exchange for glutamate. One additional possible mechanism by which boron may indirectly increase glutathione production is through the activity of enzymes such as cystathionine beta-synthase (CBS) and cystathionine gamma-lyase (CSE). Both CBS and CSE are responsible for the transport of cysteine into cells in exchange for glutamate. Additionally, CBS and CSE are enzymes involved in transsulfuration pathways, which convert homocysteine to cysteine. Since elevated levels of homocysteine have been linked to increased risk of cardiovascular disease, cognitive decline, and other health problems, lowering homocysteine levels to cysteine levels through the methionine cycle has been shown to have several potential health benefits.

Boron has shown to increase the activity of these enzymes in animal studies, which may lead to cysteine production. Boron can increase gamma-glutamyl cysteine synthetase (y-GCS) activity in liver cells, which may in turn increase glutathione production. Additionally, boron increases the expression of genes involved in glutathione metabolism, further supporting its role in enhancing glutathione production.

Of an important note, boron may enhance the stability and activity of the transcription of Nrf2, which regulates the expression of several antioxidant and detoxification enzymes, including those involved in cysteine metabolism. Activation of Nrf2 can enhance the expression of CBS and CSE and binds to DNA to promote the expression of genes involved in antioxidant and detoxification. Cystine can also activate the Nrf2 pathway by increasing the expression and activity of the enzyme cystine/glutamate transporter, which is responsible for importing cystine into the cells. This, in turn, leads to an increase in intracellular cysteine levels, which can stimulate the Nrf2 pathway and enhance cellular antioxidant defenses. In addition to cystine, other compounds such as sulforaphane, found in cruciferous vegetables and curcumin, found in turmeric, have also been shown to activate Nrf2 pathways. Accordingly, boron may increase the rate limiting amino acid cysteine in glutathione production by increasing cysteine uptake and utilization by cells by increasing expression of cysteine transporters, enhancing the activity of cysteine-metabolizing enzymes and by activating NrF2. In sum, boron provides a synergistic-like effect when included in the glutathione support compositions of the instant disclosure through a multiple of possible pathways.

Nonlimiting examples of boron sources for use with the glutathione support compositions of the invention include salts of boron. Suitable salts of boron include, but are not limited to, sodium borate, which is also known as sodium tetraborate or disodium tetraborate), boron amino acid chelate, boron ascorbate, boron aspartate, boron citrate, boron gluconate, boron glycinate, boron picolinate, calcium fructoborate, boron-β-diketonates, boron-β-thioketonates, or a combination thereof. Boron citrate is a salt of boron and citric acid, while boron glycinate is a chelated form of boron bound to the amino acid glycine. The glycine molecule acts as a carrier for the boron, helping to improve its absorption and bioavailability. It is possible that the glycine molecule may also help increase the uptake of glycine by cells. This, in turn, could potentially enhance the synthesis of collagen and other proteins that require glycine as well as support the production of glutathione. Accordingly, in various embodiments, boron glycinate is a suitable boron source.

The amount of the boron source in the glutathione support composition will vary, for example, depending on the boron source. Some boron sources include higher boron content than other boron sources. Nonetheless, in various embodiments, the glutathione support composition includes a boron source in an amount such that the glutathione support composition includes about 1 to about 500 ppm of boron. In further embodiments, the glutathione support composition includes a boron source in an amount such that the glutathione support composition includes about 1 to about 400 ppm, about 1 to about 300 ppm, about 5 to about 500 ppm, about 5 to about 400 ppm, about 5 to about 300 ppm, about 5 to about 200 ppm about 5 to about 100 ppm about 10 to about 500 ppm, about 10 to about 400 ppm, about 10 to about 300 ppm, about 10 to about 200 ppm about 10 to about 100 ppm, about 25 to about 500 ppm, about 25 to about 400 ppm, about 25 to about 300 ppm, about 25 to about 200 ppm, about 25 to about 100 ppm, or about 20 to about 80 ppm of elemental boron.

The total daily dosage amount of the boron source will vary depending on a variety of factors. Nonetheless, in various embodiments, the daily dosage amount of the boron source of (e) provides about 0.1 to about 25 mg. In further embodiments, the total daily dosage amount of the boron source provides about 0.1 to about 20 mg, about 0.1 to about 15 mg, about 0.1 to about 10 mg, about 0.5 to about 25 mg, about 0.5 to about 20 mg, about 0.5 to about 15 mg, or about 0.5 to about 10 mg.

Additional Active Ingredients

The glutathione support composition may optionally include one or more further additional active ingredients. The one or more additional active ingredients can be used instead of the boron source or in addition to a boron source.

Nonlimiting examples of further additional active ingredients include taurine, melatonin, sulforaphane, quercetin, Vitamin D3, Vitamin C, metformin, L-threonine, Rehmannia Glutinosa or an extract thereof, ashwagandha or an extract thereof, fish oil, krill oil, omega 3 fatty acids, odd-chain fatty acids (OCFA) including, but not limited to saturated C15 and C17 derivatives (pentadecanoic acid and heptadecanoic acid, respectively), kaempferol, botanical extracts, or a combination thereof.

Taurine is an amino acid that is found abundantly in the human body. It can support the immune system, calcium levels, and protect against oxidative stress. Taurine support glutathione levels in the body by increasing its synthesis and preventing breakdown. Taurine also improves the activity of enzymes that are involved in the production of glutathione, as well as enhancing the transport of glutathione into cells and provides a protective effect against glutathione breakdown by reactive oxygen species (ROS). The total amount of taurine in the glutathione support compositions, if present, will vary. Nonetheless, in various embodiments, the glutathione support composition includes about 0.1 to about 10 weight percent of taurine, based on the total weight of the glutathione support composition. In further embodiments, the glutathione support composition includes about 0.1 to about 8 weight percent, about 0.1 to about 6 weight percent, about 0.5 to about 10 weight percent, about 0.5 to about 8 weight percent, about 0.5 to about 6 weight percent, about 1 to about 10 weight percent, about 1 to about 8 weight percent, about 1 to about 6 weight percent, about 2 to about 10 weight percent, about 2 to about 8 weight percent, about 2 to about 6 weight percent (based on a total weight of the glutathione support composition).

The daily dosage amount of taurine will vary depending on a variety of factors. Nonetheless, in various embodiments, the daily dosage amount of taurine is about 1 to about 500 mg. In further embodiments, the daily dosage amount of taurine is about 1 to about 250 mg, about 1 to about 200 mg, about 1 to about 150 mg, about 5 to about 500 mg, about 5 to about 250 mg, about 5 to about 200 mg, about 5 to about 150 mg, about 10 to about 500 mg, about 10 to about 250 mg, about 10 to about 200 mg, about 10 to about 150 mg, about 25 to about 500 mg, about 25 to about 250 mg, about 25 to about 200 mg, about 25 to about 150 mg, about 50 to about 500 mg, about 50 to about 250 mg, about 50 to about 200 mg, about 50 to about 150 mg, about 100 to about 500 mg, about 100 to about 250 mg, about 100 to about 200 mg, or about 100 to about 150 mg.

Melatonin is hormone produced primarily by pineal gland and plays a role in the human body's circadian rhythms. While melatonin is best known for its role in sleep, it also has antioxidant properties in support of glutathione production. One way melatonin supports glutathione production is by reducing oxidative stress. Excess oxidative stress damages cells and contributes to various health conditions, including aging, inflammation, and chronic disease like cancer and heart disease. Melatonin helps reduce oxidative stress by scavenging reactive oxygen species and free radicals by increasing the activity of glutathione peroxidase and SOD (superoxide dismutase). By reducing oxidative stress and supporting antioxidant defense mechanisms, melatonin preserves glutathione levels in the body. Finally, melatonin appears to support glutathione production by increasing expression of genes involved in its synthesis, like Glutamate-cysteine ligase (GCL) and GSH synthetase. By upregulating these genes, melatonin may enhance the body's ability to produce and maintain optimal glutathione levels. The total amount of melatonin in the glutathione support compositions, if present, will vary. Nonetheless, in various embodiments, the glutathione support composition includes about 0.005 to about 3 weight percent of melatonin, based on the total weight of the glutathione support composition. In further embodiments, the glutathione support composition includes about 0.005 to about 2 weight percent, about 0.005 to about 1 weight percent, about 0.005 to about 0.5 weight percent, about 0.005 to about 0.3 weight percent, about 0.01 to about 3 weight percent, about 0.01 to about 2 weight percent, about 0.01 to about 1 weight percent, about 0.01 to about 0.5 weight percent, about 0.05 to about 3 weight percent, about 0.05 to about 2 weight percent, about 0.05 to about 1 weight percent, about 0.05 to about 0.5 weight percent of melatonin based on a total weight of the glutathione support composition.

The daily dosage amount of melatonin will vary depending on a variety of factors. Nonetheless, in various embodiments, the daily dosage amount of melatonin is about 0.1 to about 50 mg. In further embodiments, the daily dosage amount of melatonin is about 0.1 to about 25 mg, about 0.1 to about 10 mg, about 0.1 to about 5 mg, about 0.5 to about 50 mg, about 0.5 to about 25 mg, about 0.5 to about 20 mg, about 0.5 to about 10 mg, or about 0.5 to about 5 mg.

Sulforaphane supports glutathione production by activating NrF2 (nuclear factor erythoid 2 related factor), which is involved in regulation of antioxidant systems. When Nrf2 is activated by sulforaphane, it triggers the expression of genes involved in glutathione synthesis, such as glutamate-cysteine ligase (GCL) and GSH synthase, which can lead to increased glutathione levels. In addition to activating NrF2, sulforaphanes can enhance other enzymes for recycling, such as GSH peroxidase and GSH reductase to help maintain optimal glutathione levels by preventing depletion and promoting regeneration. The sulforaphane can be standardized with approximately 10 percent glucoraphanin.

The total amount of sulforaphane in the glutathione support compositions, if present, will vary. Nonetheless, in various embodiments, the glutathione support composition includes about 0.1 to about 10 weight percent of sulforaphane, based on the total weight of the glutathione support composition. In further embodiments, the glutathione support compositions includes about 0.1 to about 8 weight percent, about 0.1 to about 6 weight percent, about 0.1 to about 5 weight percent, about 0.5 to about 10 weight percent, about 0.5 to about 8 weight percent, about 0.1 to about 6 weight percent, about 0.1 to about 5 weight percent, about 0.5 to about 10 weight percent, about 0.5 to about 8 weight percent, about 0.5 to about 6 weight percent, about 1 to about 10 weight percent, about 1 to about 8 weight percent, about 1 to about 6 weight percent, about 2 to about 10 weight percent, about 2 to about 8 weight percent, or about 2 to about 5 weight percent of sulforaphane (based on a total weight of the glutathione support composition).

The total daily dosage amount of sulforaphane will vary depending on a variety of factors. Nonetheless, in various embodiments, the daily dosage amount of sulforaphane is from about 1 to about 1,000 mg. In further embodiments, the total daily amount of sulforaphane is about 1 to about 500 mg, about 1 to about 250 mg, about 1 to about 200 mg, about 5 to about 1,000 mg, about 5 to about 500 mg, about 5 to about 250 mg, about 5 to about 200 mg, about 10 to about 1,000 mg, about 10 to about 500 mg, about 10 to about 250 mg, about 10 to about 200 mg, about 25 to about 1,000 mg, about 25 to about 500 mg, about 25 to about 250 mg, about 25 to about 200 mg, about 50 to about 1,000 mg, about 50 to about 500 mg, about 50 to about 250 mg, about 50 to about 200 mg, or about 60 to about 140 mg.

Quercetin supports glutathione by activating Nrf2 pathway. When NrF2 is activated by quercetin, it triggers expression of genes involved in glutathione synthesis, such as GCL and GSH synthase. Quercetin also enhances glutathione synthesis and recycling similar to sulforaphane.

The total amount of quercetin in the glutathione support compositions, if present, will vary. Nonetheless, in various embodiments, the glutathione support composition includes about 0.1 to about 10 weight percent of quercetin, based on the total weight of the glutathione support composition. In further embodiments, the glutathione support composition includes about 0.1 to about 8 weight percent, about 0.1 to about 5 weight percent, about 0.1 to about 2 weight percent, about 0.5 to about 10 weight percent, about 0.5 to about 8 weight percent, about 0.5 to about 5 weight percent, about 0.5 to about 2 weight percent, about 1 to about 10 weight percent, about 1 to about 8 weight percent, about 1 to about 5 weight percent, or about 1 to about 2 weight percent (based on a total weight of the glutathione support composition).

The total daily dosage amount of quercetin will vary depending on a variety of factors. Nonetheless, in various embodiments, the daily total amount of quercetin is about 1 mg to about 1,000 mg. In further embodiments, the total daily dosage of quercetin is about 1 mg to about 500 mg, about 1 mg to about 250 mg, about 1 mg to about 100 mg, about 5 to about 1,000 mg, about 5 to about 500 mg, about 5 to about 250 mg, about 5 to about 100 mg, about 10 to about 1,000 mg, about 10 to about 500 mg, about 10 to about 250 mg, about 10 to about 100 mg, about 25 to about 1,000 mg, about 25 to about 500 mg, about 25 to about 250 mg, or about 25 to about 100 mg.

Vitamin D3 supports glutathione with several mechanism. One is via NrF2, also triggering gene expression of genes involved in glutathione synthesis, GCL and GSH synthase. Vitamin D3 also support glutathione production by increasing expression of genes involved in GSH synthesis, like Glutamate-cysteine ligase (GCL) and GSH synthetase. By upregulating these genes, vitamin D3 enhances the body's ability to produce and maintain optimal glutathione levels.

The total amount of vitamin D3 in the glutathione support compositions, if present, will vary. Nonetheless, in various embodiments, the glutathione support composition includes about 1 ppm to about 50 ppm of vitamin D3 (based on the total weight of the glutathione support composition). In further embodiments, the glutathione support composition includes about 1 ppm to about 40 ppm, about 1 ppm to about 30 ppm, about 1 ppm o about 20 ppm, about 5 ppm to about 50 ppm, about 5 ppm to about 40 ppm, about 5 ppm to about 30 ppm, or about 5 ppm to about 20 ppm of vitamin D3 (based on a total weight of the glutathione support composition).

The daily dosage amount of vitamin D3 will vary depending on a variety of factors. Nonetheless, in various embodiments, the daily dosage amount is about 1 to about 500 μg. In further embodiments, the daily dosage amount of vitamin D3 is about 1 to about 250 μg, about 1 to about 100 μg, about 1 to about 50 μg, about 5 to about 500 μg, about 5 to about 250 μg, about 5 to about 100 μg, about 5 to about 50 μg, about 10 to about 500 μg, about 10 to about 250 μg, about 10 to about 100 μg, or about 10 to about 50 μg.

In some embodiments, a mixture of vitamin D3 and vitamin k2 is used. In some aspects, the vitamin k2 is used in an amount of about 20 mcg to about 75 μg and the vitamin D3 may constitute the remainder of the daily dosage amount. For example, vitamin K2 may be included in the mixture in an amount of about 30 μg to about 55 μg. In this aspect, the daily dosage amount of the mixture will likewise vary depending on a variety of factors. Nonetheless, in various embodiments, the daily dosage amount of the mixture is about 1 to about 500 μg. In further embodiments, the daily dosage amount of the mixture is about 1 to about 250 μg, about 1 to about 100 μg, about 1 to about 50 μg, about 5 to about 500 μg, about 5 to about 250 μg, about 5 to about 100 μg, about 5 to about 50 μg, about 10 to about 500 μg, about 10 to about 250 μg, about 10 to about 100 μg, or about 10 to about 50 μg.

Vitamin C, aka ascorbic acid, is a water-soluble vitamin that acts as a potent antioxidant. The total amount of vitamin C in the glutathione support compositions, if present, will vary. Nonetheless, in various embodiments, the glutathione support composition includes about 0.1 to about 15 weight percent of vitamin C (based on the total weight of the glutathione support composition). In further embodiments, the glutathione support composition includes about 0.1 to about 12 weight percent, about 0.1 to about 10 weight percent, about 0.1 to about 5 weight percent, about 0.5 to about 15 weight percent, about 0.5 to about 12 weight percent, about 0.5 to about 10 weight percent, about 0.5 to about 5 weight percent, about 1 to about 15 weight percent, about 1 to about 12 weight percent, about 1 to about 10 weight percent, about 1 to about 5 weight percent, about 2 to about 15 weight percent, about 2 to about 12 weight percent, about 2 to about 10 weight percent, about 2 to about 5 weight percent, about 5 to about 15 weight percent, about 5 to about 12 weight percent, or about 5 to about 10 weight percent if vitamin C (based on a total weight of the glutathione support composition).

The daily dosage amount of vitamin C will vary depending on a variety of factors. Nonetheless, in various embodiments, the daily dosage amount of vitamin C is about 10 mg to about 1,000 mg. In further embodiments, the daily dosage amount of vitamin C is about 10 to about 750 mg, about 10 to about 500 mg, about 10 to about 250 mg, about 50 to about 1,000 mg, about 50 to about 500 mg, about 50 to about 250 mg, about 100 to about 1,000 mg, about 100 to about 500 mg, or about 100 to about 250 mg.

Metformin supports glutathione production is by activating the AMPK (adenosine monophosphate activated protein kinase) pathway. Activation of AMPK pathway increases expression of genes involved in glutathione synthesis, such as GCL and GSH synthase, leading to an increase in GSH levels.

The total amount of metformin in the glutathione support compositions, if present, will vary. Nonetheless, in various embodiments, the glutathione support composition includes about 0.1 to about 15 weight percent of metformin (based on the total weight of the glutathione support composition). In further embodiments, the glutathione support composition includes about 0.1 to about 12 weight percent, about 0.1 to about 10 weight percent, about 0.1 to about 5 weight percent, about 0.5 to about 15 weight percent, about 0.5 to about 12 weight percent, about 0.5 to about 10 weight percent, about 0.5 to about 5 weight percent, about 1 to about 15 weight percent, about 1 to about 12 weight percent, about 1 to about 10 weight percent, about 1 to about 5 weight percent, about 2 to about 15 weight percent, about 2 to about 12 weight percent, about 2 to about 10 weight percent, about 2 to about 5 weight percent, about 5 to about 15 weight percent, about 5 to about 12 weight percent, or about 5 to about 10 weight percent of metformin (based on a total weight of the glutathione support composition).

The daily dosage amount of metformin will vary depending on a variety of factors. Nonetheless, in various embodiments, the daily dosage amount of metformin is about 10 mg to about 1,000 mg. In further embodiments, the daily dosage amount of metformin is about 10 to about 750 mg, about 10 to about 500 mg, about 10 to about 250 mg, about 50 to about 1,000 mg, about 50 to about 500 mg, about 50 to about 250 mg, about 100 to about 1,000 mg, about 100 to about 500 mg, or about 100 to about 250 mg.

L-threonine is an essential amino acid that plays a crucial role in protein synthesis in support of glutathione production in vivo. L-threonine supports glutathione production by serving as a precursor for synthesis of cysteine. L-threonine is converted to glycine, which is converted to cysteine via enzymatic reactions. Cysteine residues are included as part of glutathione. The total amount of L-threonine in the glutathione support compositions, if present, will vary. Nonetheless, in various embodiments, the glutathione support composition includes about 1 to about 30 weight percent of L-threonine (based on the total weight of the glutathione support composition). In further embodiments, the glutathione support composition includes about 1 to about 25 weight percent, about 1 to about 20 weight percent, about 1 to about 15 weight percent, about 1 to about 10 weight percent, about 2 to about 30 weight percent, about 2 to about 25 weight percent, about 2 to about 20 weight percent, about 2 to about 15 weight percent, about to about 10 weight percent, about 5 to about 30 weight percent, about 5 to about 25 weight percent, about 5 to about 20 weight percent, about 5 to about 15 weight percent, about 5 to about 10 weight percent, about 10 to about 30 weight percent, about 10 to about 25 weight percent, or about 10 to about 20 weight percent (based on a total weight of the glutathione support composition).

The daily dosage amount of L-threonine will vary depending on a variety of factors. Nonetheless, in various embodiments, the daily dosage amount of L-threonine is about 10 mg to about 2,000 mg. In further embodiments, the daily dosage amount of L-threonine is about 10 to about 1,500 mg, about 10 to about 1,000 mg, about 10 to about 500 mg, about 50 to about 2,000 mg, about 50 to about 1,000 mg, about 50 to about 500 mg, about 100 to about 2,000 mg, about 100 to about 1,000 mg, about 100 to about 500 mg, about 250 to about 2,000 mg, about 250 to about 1,500 mg, about 250 to about 1,000 mg, or about 250 to about 500 mg.

L-theanine, also known as L-γ-glutamylethylamide and N5-ethyl-L-glutamine, is an amino acid analogue of the proteinogenic amino acids L-glutamate and L-glutamine and is found primarily in particular plant and fungal species. The total amount of L-theanine in the glutathione support compositions, if present, will vary. Nonetheless, in various embodiments, the glutathione support composition includes about 1 to about 15 weight percent of L-theanine (based on the total weight of the glutathione support composition). In further embodiments, the glutathione support composition includes about 1 to about 12 weight percent, about 1 to about 10 weight percent, about 1 to about 5 weight percent, about 2 to about 15 weight percent, about 2 to about 12 weight percent, about 2 to about 10 weight percent, about 2 to about 5 weight percent, about 5 to about 15 weight percent, about 5 to about 12 weight percent, or about 5 to about 10 weight percent of L-theanine (based on a total weight of the glutathione support composition).

The daily dosage amount of L-theanine will vary depending on a variety of factors. Nonetheless, in various embodiments, the daily dosage amount of L-theanine is about 10 mg to about 1,000 mg. In further embodiments, the daily dosage amount of L-theanine is about 10 to about 800 mg, about 10 to about 500 mg, about 10 to about 300 mg, about 10 to about 200 mg, about 50 to about 1,000 mg, about 50 to about 800 mg, about 50 to about 500 mg, about 50 to about 300 mg, about 50 to about 200 mg, about 100 to about 1,000 mg, about 100 to about 800 mg, about 100 to about 500 mg, about 100 to about 300 mg, about 100 to about 200 mg.

Turmeric is a flowering plant, Curcuma longa of the ginger family, Zingiberaceae and can be useful for reducing inflammation. Curcumin is a bright yellow chemical produced by plants of the Curcuma longa species. It is the principal curcuminoid of turmeric (Curcuma longa).

The total amount of turmeric in the glutathione support compositions, if present, will vary. Nonetheless, in various embodiments, the glutathione support composition includes about 1 to about 25 weight percent of turmeric (based on the total weight of the glutathione support composition). In further embodiments, the glutathione support composition includes about 1 to about 20 weight percent, about 1 to about 15 weight percent, about 1 to about 12 weight percent, about 1 to about 10 weight percent, about 1 to about 5 weight percent, about 2 to about 25 weight percent, about 2 to about 20 weight percent, about 2 to about 15 weight percent, about 2 to about 12 weight percent, about 2 to about 10 weight percent, about 2 to about 5 weight percent, about 5 to about 25 weight percent, about 5 to about 20 weight percent, about 5 to about 15 weight percent, about 5 to about 12 weight percent, or about 5 to about 10 weight percent of turmeric (based on a total weight of the glutathione support composition).

The daily dosage amount of turmeric will vary depending on a variety of factors. Nonetheless, in various embodiments, the daily dosage amount of turmeric is about 100 mg to about 2,000 mg. In further embodiments, the daily dosage amount of turmeric is about 101 to about 1,800 mg, about 100 to about 1,500 mg, about 100 to about 1,200 mg, about 100 to about 1,000 mg, about 100 to about 800 mg, about 100 to about 500 mg, about 250 to about 2,000 mg, about 250 to about 1,800 mg, about 250 to about 1,500 mg, about 250 to about 1,200 mg, about 250 to about 1,000 mg, about 250 to about 800 mg, about 250 to about 500 mg.

Ashwaganda, aka Withania somnifera, is an adaptogenic herb shown to exhibit antioxidant and immunomodulatory properties in support of glutathione production in vivo. Ashwaganda reduces oxidative stress and inflammation in the body and increases expression of genes involved in glutathione synthesis such as GCL and GSH synthase. The ashwaganda may be a root extract, a root powder, or a combination thereof.

The total amount of ashwaganda in the glutathione support compositions, if present, will vary. Nonetheless, in various embodiments, the glutathione support composition includes about 1 to about 30 weight percent of ashwaganda (based on the total weight of the glutathione support composition). In further embodiments, the glutathione support composition includes about 1 to about 25 weight percent, about 1 to about 20 weight percent, about 1 to about 15 weight percent, about 1 to about 10 weight percent, about 2 to about 30 weight percent, about 2 to about 25 weight percent, about 2 to about 20 weight percent, about 2 to about 15 weight percent, about to about 10 weight percent, about 5 to about 30 weight percent, about 5 to about 25 weight percent, about 5 to about 20 weight percent, about 5 to about 15 weight percent, about 5 to about 10 weight percent, about 10 to about 30 weight percent, about 10 to about 25 weight percent, or about 10 to about 20 weight percent of ashwaganda (based on a total weight of the glutathione support composition).

The daily dosage amount of the ashwaganda will vary depending on a variety of factors. Nonetheless, in various embodiments, the daily dosage amount of the ashwaganda (root extract, root powder, or combination thereof) is about 10 to about 2,000 mg. In further embodiments, the daily dosage amount of the ashwaganda is about 10 to about 1,500 mg, about 10 to about 1,000 mg, about 10 to about 500 mg, about 25 to about 2,000 mg, about 25 to about 1,000 mg, about 25 to about 500 mg, about 50 to about 2,000 mg, about 50 to about 1,500 mg, about 50 to about 1,000 mg, about 50 to about 500 mg, about 100 to about 2,000 mg, about 100 to about 1,500 mg, about 100 to about 1,000 mg, or about 100 to about 500 mg.

Fish oil, krill oil, omega-3-fatty acids, and/or OCFA also provide antioxidant and other health benefits to humans and can synergistically enhance the health promoting effects of the glutathione support compositions.

The total amount of fish oil, krill oil, and/or omega-3-fatty acids in the glutathione support compositions, if present, will vary. Nonetheless, in various embodiments, the glutathione support composition includes about 1 to about 30 weight percent of fish oil, krill oil, and/or omega-3-fatty acids (based on the total weight of the glutathione support composition). In further embodiments, the glutathione support composition includes about 1 to about 25 weight percent, about 1 to about 20 weight percent, about 1 to about 15 weight percent, about 1 to about 10 weight percent, about 2 to about 30 weight percent, about 2 to about 25 weight percent, about 2 to about 20 weight percent, about 2 to about 15 weight percent, about to about 10 weight percent, about 5 to about 30 weight percent, about 5 to about 25 weight percent, about 5 to about 20 weight percent, about 5 to about 15 weight percent, about 5 to about 10 weight percent, about 10 to about 30 weight percent, about 10 to about 25 weight percent, or about 10 to about 20 weight percent of fish oil, krill oil, and/or omega-3-fatty acids (based on a total weight of the glutathione support composition).

The daily dosage amount of fish oil, krill oil, and/or omega-3-fatty acids will vary depending on a variety of factors. Nonetheless, in various embodiments, the daily dosage amount is from about 5 to about 2,000 mg. In further embodiments, the daily dosage amount of the fish oil, krill oil, and/or omega-3-fatty acids is about 5 to about 1,500 mg, about 5 to about 1,000 mg, about 5 to about 500 mg, about 25 to about 2,000 mg, about 25 to about 1,500 mg, about 25 to about 1,000 mg, about 25 to about 500 mg, about 100 to about 2,000 mg, about 100 to about 1,500 mg, about 100 to about 1,000 mg, about 100 to about 500 mg, about 250 to about 2,000 mg, about 250 to about 1,500 mg, about 250 to about 1,000 mg, about 250 to about 500 mg.

The glutathione support compositions may optionally include one or more botanical extracts. Nonlimiting examples include bamboo extract, for example, bamboo extract rich in minerals (e.g., silica) essential for collagen synthesis, Gotu Kola, grape seed extract, in particular grape seed extracts rich in antioxidants called oligomeric proanthocyanidins (OPCs), pomegranate extract, and pomegranate skin extract, which is often rich in antioxidant ellagitannins, aloe vera, contains acemannan, green tea extract, which is rich in antioxidants called catechins, or a combination thereof.

Finally, the glutathione support composition may include one or more enzymes, for example, one or more digestive enzymes. For example, the glutathione support composition may include one or more amylases, lipases, proteases, lactases, sucrases, and the like.

Optional Ingredients

The glutathione support compositions, as optional ingredients, may include pharmaceutically or nutraceutical acceptable carriers and diluents, which are available in the art. However, the proteins or peptides of the collagen source, in addition to their glutathione potentiating and other health benefits, can simultaneously serve as a carrier or filler in addition to their role in glutathione synthesis. For example, gelatin can be used to protect other active ingredients in the glutathione support composition, can add bulk to tablets, and can function as a thickener for liquid or edible products. In addition, the glutathione support compositions themselves can be used as a filler in other nutraceuticals or pharmaceutical products, to supplement and enhance the product, for example, by replacing lactose or other traditional fillers used in industry.

Specific nonlimiting examples of the carriers and/or diluents include water and physiologically acceptable buffered saline solutions such as phosphate buffered saline solutions pH 7.0-8.0. Suitable carriers include, but are not limited to sterile water, salt solutions (such as Ringer's solution), alcohols, polyethylene glycols, gelatin, carbohydrates such as lactose, amylose or starch, magnesium stearate, talc, silicic acid, viscous paraffin, fatty acid esters, hydroxymethyl cellulose, polyvinylpyrrolidone, and the like. The glutathione support compositions can be mixed with auxiliary agents, e.g., lubricants, stabilizers, wetting agents, emulsifiers, solubilizing agents, salts for influencing osmotic pressure, buffers, thickeners, surface active agents, neutral or cationic lipids, lipid complexes, liposomes, penetration enhancers, coloring, and/or aromatic substances and the like which do not deleteriously react with the active compounds of the glutathione support compositions.

In some embodiments, the glutathione support compositions are in an orally available dosage form, i.e., a dosage form appropriate for oral consumption. However, the glutathione support compositions may be in any form which allows for the composition to be administered to a patient. The glutathione support compositions are formulated so the active ingredients are bioavailable upon administration of the composition to a subject. Compositions that will be administered to a subject may take the form of one or more dosage units. For example, a dosage unit may include one or more tablets, one or more capsules, a predetermined amount of powder or granules, a sachet, a gummy or other edible form, a drink including a carbonated drink, a syrup, etc. A container is typically used to hold a plurality of dosage units, for example, the container holds a powder, granules, capsules, tablets, a gummies or other edible forms, a drink, a syrup, and the like.

For oral administration, an excipient and/or binder may be present. Examples are sucrose, kaolin, glycerin, starch dextrin, sodium alginate, carboxymethylcellulose, and ethyl cellulose. Coloring and/or flavoring agents may be present. A coating shell may be employed, applying common membranes used for microencapsulation and suitable for the microencapsulation of live probiotic organisms include biodegradable synthetic “polymers” such as polylactide, polyglycolic acid, and polyanhydride. Nonlimiting examples of “polymers” include alginate-polylysine-alginate (APA), alginate-polymethylene-co-guanidine-alginate (A-PMCG-A), hydroymethylacrylate-methyl methacrylate (HEMA-MMA), Multilayered HEMA-MMA-MAA, polyacrylonitrile-vinylchloride (PAN-PVC), acrylonitrile/sodium methallylsulfonate (AN-69), polyethylene glycol/poly pentamethylcyclopentasiloxane/polydimethylsiloxane (PEG/PD/PDMS), poly N,N dimethyl acrylamide (PDMAAm), Siliceous encapsulates and cellulose sulphate/sodium alginate/polymethylene-co-guanidine (CS/A/PMCG). Other materials that are useful include, without limitation, cellulose acetate phthalate, calcium alginate and k-carrageenan-Locust bean gum gel beads, gellan-xanthan beads, poly(lactide-co-glycolides), carrageenan, starch poly-anhydrides, starch polymethacrylates, polyamino acids, enteric coating polymers, or a combination thereof.

The glutathione support composition may include natural and/or artificial sweeteners. In some embodiments, the sweetener may be trehalose, aspartame, acesulfame potassium, aspartame-acesulfame salt, advantame, neotame, neohesperidin, saccharin, sucralose, stevia, or combinations thereof.

A liquid glutathione support composition as used herein, whether in the form of a solution, suspension or other like form, may include one or more of the following adjuvants: diluents such as water, fixed oils such as synthetic mono or diglycerides that may serve as the solvent or suspending medium, polyethylene glycols, glycerin, propylene glycol or other solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; preservatives to ensure safety and shelf-life; and sweeteners including natural and/or artificial sweeteners.

In some aspects, the glutathione support compositions include:
(a) about 30 to about 80 weight percent of a collagen source having proteins or peptides including at least 5 percent proline residues, at least 5 percent hydroxyproline residues, and at least 20 percent glycine residues (based on a total weight average of the proteins or peptides);
(b) about 15 to about 50 weight percent of a glutamate source;
(c) about 5 to about 25 weight percent of a cysteine source, where a total amount of (a), (b), and (c) constitute about 50 to about 99.9 weight percent of the composition;
(d) a selenium source; and
(e) optionally, a boron source and/or one or more additional active ingredient.

The present disclosure also relates to glutathione support compositions that includes about 50 to about 99.99 weight percent, about 60 to about 99.99 weight percent, about 65 to about 99.99 weight percent, about 70 to about 99.99 weight percent, about 75 to about 99.99 weight percent, about 80 to about 99.99 weight percent, about 85 to about 99.99 weight percent, about 90 to about 99.99 weight percent, or about 95 to about 99.99 weight percent of a combination of (a), (b), and (c):
(a) a collagen source;
(b) a glutamate source; and
(c) a cysteine source (relative to a total weight of the glutathione support composition).

In some embodiments, the glutathione support composition includes about 30 to about 80 weight percent of the collagen source (based on the total weight of the glutathione support composition). In on aspect, the glutathione support composition includes about 35 to about 65 weight percent of the collagen source (based on the total weight of the glutathione support composition). In another aspect, the glutathione support composition includes about 40 to about 60 weight percent of the collagen source (based on the total weight of the glutathione support composition).

In other embodiments, the glutathione support composition includes about 10 to about 50 weight percent of the glutamate source (based on the total weight of the glutathione support composition). In one aspect, the glutamate source is present in an amount of about 25 to about 50 weight percent (based on the total weight of the glutathione support composition). In another aspect, the glutamate source is present in an amount of about 20 to about 45 weight percent (based on the total weight of the glutathione support composition).

In yet other embodiments, the glutathione support composition includes about 5 to about 30 weight percent of the cysteine source (based on the total weight of the glutathione support composition). In one aspect, the cysteine source is present in an amount of about 10 to about 30 weight percent (based on the total weight of the glutathione support composition). In another aspect, the cysteine source is present in an amount of about 15 to about 30 weight percent (based on the total weight of the glutathione support composition).

In further embodiments, (a) and (b) in either formulation above, i.e., collagen source and the glutamate source, are in a molar ratio of about 1:5 to about 5:1, about 1:4 to about 4:1, about 1:3 to about 3:1, about 1:2 to about 2:1, or about 1:1 ((a):(b)). Furthermore, (a) and (c), i.e., the glycine and cysteine sources, may be in a molar ratio of about 10:1 to about 1:1, about 8:1 to about 1:1, about 5:1 to about 1:1, about 4:1 to about 1:1, about 3:1 to about 1:1, or about 2:1 ((a):(c)). Finally, (b) and (c), i.e., the glutamate and cysteine sources, may be in a molar ratio of about 10:1 to about 1:1, about 8:1 to about 1:1, about 5:1 to about 1:1, about 4:1 to about 1:1, about 3:1 to about 1:1, or about 2:1 ((b):(c)). For example, the molar ratio of (b):(c) may be about 1:1 to about 2.5:1. In some embodiments, (a), (b), and (c), are in a molar ratio of about 2:2:1 ((a):(b):(c)). In one embodiment, the molar ratio of the collagen source to the glutamate source and the cysteine source (a):((b)+(c)) is about 0.8:1 to about 1.1:1.

### Methods of Use and Administration

The glutathione support compositions of the instant disclosure are useful in potentiating, increasing, and maintaining intracellular glutathione levels. Such methods typically include administering, preferably orally administering, the glutathione support composition to an individual. As used herein, the term “individual” refers to an animal, preferably a mammal, and even more preferably, a human. The terms “host,” “subject,” and “patient” are interchangeable with the term “individual.”

The methods are particularly useful for older adults, as intracellular glutathione levels tend to decrease naturally with age. The methods are also particularly useful for individuals that produce less than normal amounts of glutathione including individual suffering from one or more genetic mutations that impede the ability of the individual's body to naturally produce glutathione. Nonlimiting examples of genetic mutations include mutations of Glutathione peroxidase 1 gene (GPX1 gene), Glutathione S-transferase P gene (GSTP1 gene), Glutathione synthetase gene (GS gene), Glutathione S-transferase gene (GST gene), Glutathione 5-transferase theta-1 gene (GSTT1 gene), and the methylenetetrahydrofolatereductase gene (MTHFR gene).

Examples of genetic mutations in the Glutathione S-transferase gene (GST gene) include Ile105Val (rs1695) of the glutathione S-transferase P1, i.e., a mutation causing amino acid isoleucine 105 changed to a valine (Ile105Val) or amino acid alanine 114 changed to a valine (Ala114Val). Examples of genetic mutations in the Glutathione S-transferase theta-1 gene (GSTT1) gene include a null/present polymorphism. Examples of mutations in the glutathione S-transferase (GSTM1) gene include a null/present polymorphism. Examples of mutations in the methylenetetrahydrofolatereductase gene (MTHFR gene) include MTHFR C677T and A1298C.

The glutathione support composition is preferably administered daily, and is preferably orally administered, although other routes of administration are envisioned. The form of the glutathione support composition will influence what, if any, carriers are included in the glutathione support composition. In various embodiments, the glutathione support compositions may be administered intravenously, by injection, with a nebulizer, intranasally, by inhalation, with a gastrostomy tube, or as a suppository.

Solid oral dosage forms for oral administration can include capsules, sustained-release capsules, tablets, sustained release tablets, chewable tablets, sublingual tablets, effervescent tablets, pills, powders, liquids, granules, gummies, and gels. In such solid dosage forms, the composition can include at least one inert diluent such as sucrose, lactose or starch, lubricating agents such as magnesium stearate, and buffering agents (in the case of capsules, tablets, effervescent tablets, and pills). Soft gelatin capsules can be prepared to contain a mixture of the glutathione support composition and vegetable oil. Hard gelatin capsules may contain granules of the glutathione support composition in combination with a carrier such as lactose, saccharose, sorbitol, mannitol, potato starch, corn starch, amylopectin, cellulose derivatives of gelatin, and combinations thereof. In some embodiments, the oral dosage form is an ingredient for foodstuff, for example, an ingredient for bread.

It is useful to administer the glutathione support composition before treatment with an antibiotic. However, it is also useful to begin treatment with a glutathione support composition at the same time treatment begins with an antibiotic. Indeed, benefits from the glutathione support composition of the present disclosure are also noticeable when treatment with the glutathione support composition begins after treatment with the antibiotic has begun. In this aspect, the glutathione support composition can be administered separately from the antibiotic or together with the antibiotic. When the glutathione support composition and one or more antibiotics are administered together, it can be useful if the glutathione support composition and the antibiotic are formulated into a single dosage form, e.g., any of the oral or liquid dosages forms discussed previously. In various embodiments, the glutathione support composition can be used as a filler or part of a filler in the manufacture of a dosage form, for example, a tablet. In situation where it is not practical or desired to include the glutathione support composition in the same dosage form as the antibiotic, the glutathione support composition and the antibiotic can be supplied in a kit, wherein the kit includes one or more doses of the glutathione support composition and one or more doses of an antibiotic.

Daily administration may include a single administration once per day, wherein the single administration provides a total daily dosage amount. Daily administration may also include multiple administrations throughout the day. In particular, an amount of the glutathione support composition can be administered throughout the day until a total daily dosage amount is administered. For example, in some embodiments, a first part of the total daily dosage amount may be administered at a first time in the day and the second, remaining part of the total daily dosage may be administered at a second time in the day. In this aspect, the first and second times may be about 4 to 8 hours apart. In addition, the first and second times may be morning and evening, respectively. In other embodiments, the total daily dosage amount of the glutathione support composition can be split into more than 2 parts, each part being administered at separate times throughout the day until the total daily dosage amount is reached.

In various embodiments, the glutathione support composition is administered to the individual daily, for example, for at least 3 consecutive days, at least 5 consecutive days, at least 7 consecutive days, at least 10 consecutive days, at least 14 consecutive days, at least 20 consecutive days, at least 28 consecutive days or longer.

Dosing

The glutathione support compositions are typically administered in a particular dosage amount. Dosage amounts will vary based on a variety of factors, for example, the frequency and/or length of the dosing, the weight, age, and/or sex of the individual being dosed, the disease state, nutritional needs, and the form of the glutathione support composition (e.g., powder, tablet, liquid, capsules, edible, etc.). Dosing can be administered as a preventative health measure and part of a wellness and/or longevity protocol.

As recognized by those in the art, larger doses are administered to adults and smaller doses are administered to children, especially small children. The form of the glutathione support composition can also influence dosing. The glutathione support compositions can be in a variety of different forms. For example, the compositions can in the form of a powder, a liquid, a tablet, a capsule, an edible, a gummy, a beverage, a carbonated beverage, in a sachet, a chewable or effervescent tablet etc. If the glutathione support composition is administered in a beverage, the dosage amount for the beverage will most likely be higher than when the glutathione support composition is administered as a powder, tablet, capsules, etc., due to the inclusion of additional ingredients constituting the beverage (for example, water, flavorings, sweeteners, etc.).

Consumers tend to prefer oral administration over other types of administration, for example, injection, infusion, sublingual, nasal, rectal, vaginal, subcutaneous, intramuscular, etc. Nonetheless, the fact that oral administration of the glutathione support composition is preferred does not suggest that other types of administration are not able to be used in accordance with the methods of the present disclosure. In fact, it may be useful in some contexts to administer the glutathione support composition by injection, infusion, sublingually, nasally, rectally, vaginally, subcutaneously, intramuscularly, and the like. The form of the glutathione support composition will influence what, if any, carriers are included in the glutathione support composition. In various embodiments, the glutathione support compositions may be administered intravenously, by injection, with a nebulizer, intranasally, by inhalation, with a gastrostomy tube, or as a suppository. Orally administered compositions, for example, may be in the form of powder, tablets, capsules, a food-like product (e.g., gummies), or as a liquid. Accordingly, administration of the glutathione support composition is certainly not limited to oral administration even though consumers tend to prefer oral administration.

The glutathione support compositions can be administered daily, multiple times per day, or less frequently than daily administration. In a preferred embodiment, a daily dosage amount of glutathione support composition is administered for a number of consecutive days. For example, for at least 3 consecutive days, at least 7 consecutive days, at least 14 consecutive days, at least 30 consecutive days, for at least 1 month daily, for at least 2 months daily, for at least 6 months daily, or longer.

A daily dosage amount can be administered in a single daily dose or can be administered in smaller amounts multiple times throughout the day, for example, 2, 3, or 4 times per day. For purposes of illustration only, a 2500 mg daily dose of the glutathione support composition may be administered one time per day. Alternatively, the 2500 mg daily dose can be attained by administering 1250 mg of the glutathione support composition in the morning and by administering another 1250 mg of the glutathione support composition in the evening. Alternatively, when an individual has higher risk of oxidative stress, the glutathione support compositions of the instant disclosure may be administered a 2500 mg dose twice daily.

In various embodiments, the daily dosage amount of the glutathione support composition comprises:
(a) about 100 to about 4,000 mg of a collagen source;
(b) about 400 to about 2,000 mg of a glutamate source;
(c) about 100 to about 2,000 of a cysteine source;
(d) a selenium source, preferably a selenium source in an amount providing about 1 to about 50 μg of elemental selenium;
(e) optionally, a boron source in an amount providing about 0.1 to about 50 mg of elemental boron; and
(f) optionally, one or more additional active ingredients.

In some embodiments, the additional active ingredient(s) includes a zinc source, methylfolate, methyl-B₁₂, quercetin, sulforaphane, methionine, vitamin D, vitamin C, L-threonine, taurine, melatonin, ashwagandha, fisetin, 1-NMA, NAD+, or kaempferol (or other flavonoid antioxidants), or a combination thereof. The dosage amount for each of the one or more additional active ingredients will vary depending on the ingredient. In any event, the one or more additional active ingredients are in amount that is physiologically safe and effective.

The daily dosage amount of the one or more proteins or peptides of (a) will vary depending on a variety of factors, discussed above (e.g., age, sex, medical condition, etc.). Nonetheless, in various embodiments, the daily dosage amount of the collagen source may be from about 100 mg to about 4,000 mg. In further embodiments, the daily dosage amount is from about 200 to about 4,000 mg, about 500 to about 4,000 mg, about 1,000 to about 4,000 mg, about 100 to about 3,000 mg, about 200 to about 3,000 mg, about 500 to about 3,000 mg, about 1,000 to about 3,000 mg, about 100 to about 2,000 mg, about 200 to about 2,000 mg, about 500 to about 2,000 mg, about 1,000 to about 2,000 mg, about 100 to about 1,500 mg, about 200 to about 1,500 mg, about 500 to about 1,500 gm, or about 1,000 to about 1,500 mg.

The daily dosage amount of the glutamate source will vary depending on a variety of factors. Nonetheless, in various embodiments, the daily dosage amount of the glutamate source may be from about 100 mg to about 2,000 mg. In further embodiments, the daily dosage amount is from about 200 to about 2,000 mg, about 500 to about 2,000 mg, about 100 to about 1,500 mg, about 200 to about 1,500 mg, about 500 to about 1,500 mg, about 100 to about 1,200, about 200 to about 1,200 mg, about 500 to about 1,200 mg, about 100 to about 1,000 mg, about 200 to about 1,000 mg, about 500 to about 1,000 mg, or about 600 to about 1,200 mg.

The daily dosage amount of the cysteine source will vary depending on a variety of factors. Nonetheless, in various embodiments, the daily dosage amount of the cysteine source may be from about 100 mg to about 2,000 mg. In further embodiments, the daily dosage amount is from about 200 to about 2,000 mg, about 500 to about 2,000 mg, about 100 to about 1,500 mg, about 200 to about 1,500 mg, about 500 to about 1,500 mg, about 100 to about 1,200, about 200 to about 1,200 mg, about 500 to about 1,200 mg, about 100 to about 1,000 mg, about 200 to about 1,000 mg, about 100 to about 800 mg, or about 100 to about 500 mg.

The daily dosage amount of the selenium source will vary depending on a variety of factors. Nonetheless, in various embodiments, the daily dosage amount of the selenium source provides about 1 to about 100 μg of elemental selenium. In further embodiments, the daily dosage amount of the selenium source of (d) provides 1 to about 80 μg, about 1 to about 60 μg, about 1 to about 50 ng, about 1 to about 25 ng, about to about 20 ng, about 2 to about 100 ng, about 2 to about 80 ng, about 2 to about 60 ng, about 2 to about 50 ng, about 2 to about 25 ng, about 2 to about 25 ng, about 2 to about 20 ng, about 5 to about 100 ng, about 5 to about 80 ng, about 5 to about 60 ng, about 5 to about 50 ng, about 5 to about 25 ng, or about 5 to about 20 μg.

The daily dosage amount of the boron source will vary depending on a variety of factors. Nonetheless, in various embodiments, the daily dosage amount of the boron source provides about 0.1 to about 25 mg. In further embodiments, the total daily dose of the boron source provides about 0.1 to about 20 mg, about 0.1 to about 15 mg, about 0.1 to about 10 mg, about 0.5 to about 25 mg, about 0.5 to about 20 mg, about 0.5 to about 15 mg, or about 0.5 to about 10 mg.

The daily dosage amount of taurine will vary depending on a variety of factors. Nonetheless, in various embodiments, the daily dosage amount of taurine is about 1 to about 500 mg. In further embodiments, the daily dosage amount of taurine is about 1 to about 250 mg, about 1 to about 200 mg, about 1 to about 150 mg, about 5 to about 500 mg, about 5 to about 250 mg, about 5 to about 200 mg, about 5 to about 150 mg, about 10 to about 500 mg, about 10 to about 250 mg, about 10 to about 200 mg, about 10 to about 150 mg, about 25 to about 500 mg, about 25 to about 250 mg, about 25 to about 200 mg, about 25 to about 150 mg, about 50 to about 500 mg, about 50 to about 250 mg, about 50 to about 200 mg, about 50 to about 150 mg, about 100 to about 500 mg, about 100 to about 250 mg, about 100 to about 200 mg, or about 100 to about 150 mg.

The daily dosage amount of melatonin will vary depending on a variety of factors. Nonetheless, in various embodiments, the total daily dose of melatonin is about 0.1 to about 50 mg. In further embodiments, the total daily dose of melatonin is about 0.1 to about 25 mg, about 0.1 to about 10 mg, about 0.1 to about 5 mg, about 0.5 to about 50 mg, about 0.5 to about 25 mg, about 0.5 to about 20 mg, about 0.5 to about 10 mg, or about 0.5 to about 5 mg.

The daily dosage amount of sulforaphane will vary depending on a variety of factors. Nonetheless, in various embodiments, the total daily dose of sulforaphane is from about 1 to about 1,000 mg. In further embodiments, the total daily amount of sulforaphane is about 1 to about 500 mg, about 1 to about 250 mg, about 1 to about 200 mg, about 5 to about 1,000 mg, about 5 to about 500 mg, about 5 to about 250 mg, about 5 to about 200 mg, about 10 to about 1,000 mg, about 10 to about 500 mg, about 10 to about 250 mg, about 10 to about 200 mg, about 25 to about 1,000 mg, about 25 to about 500 mg, about 25 to about 250 mg, about 25 to about 200 mg, about 50 to about 1,000 mg, about 50 to about 500 mg, about 50 to about 250 mg, about 50 to about 200 mg, or about 60 to about 140 mg.

The daily dosage amount of quercetin will vary depending on a variety of factors. Nonetheless, in various embodiments, the daily total amount of quercetin is about 1 mg to about 1,000 mg. In further embodiments, the total daily dosage of quercetin is about 1 mg to about 500 mg, about 1 mg to about 250 mg, about 1 mg to about 100 mg, about 5 to about 1,000 mg, about 5 to about 500 mg, about 5 to about 250 mg, about 5 to about 100 mg, about 10 to about 1,000 mg, about 10 to about 500 mg, about 10 to about 250 mg, about 10 to about 100 mg, about 25 to about 1,000 mg, about 25 to about 500 mg, about 25 to about 250 mg, or about 25 to about 100 mg.

The daily dose amount of vitamin D3 will vary depending on a variety of factors. Nonetheless, in various embodiments, the total daily dose is about 1 to about 500 μg. In further embodiments, the total daily dose of vitamin D3 is about 1 to about 250 μg, about 1 to about 100 μg, about 1 to about 50 μg, about 5 to about 500 μg, about 5 to about 250 μg, about 5 to about 100 μg, about 5 to about 50 μg, about 10 to about 500 μg, about 10 to about 250 μg, about 10 to about 100 μg, or about 10 to about 50 μg.

The daily dosage amount of vitamin C will vary depending on a variety of factors. Nonetheless, in various embodiments, the total daily dose of vitamin C is about 10 mg to about 1,000 mg. In further embodiments, the daily dosage amount of vitamin C is about 10 to about 750 mg, about 10 to about 500 mg, about 10 to about 250 mg, about 50 to about 1,000 mg, about 50 to about 500 mg, about 50 to about 250 mg, about 100 to about 1,000 mg, about 100 to about 500 mg, or about 100 to about 250 mg.

The daily dosage amount of metformin will vary depending on a variety of factors. Nonetheless, in various embodiments, the daily dosage amount of metformin is about 10 mg to about 1,000 mg. In further embodiments, the total daily dose of metformin is about 10 to about 750 mg, about 10 to about 500 mg, about 10 to about 250 mg, about 50 to about 1,000 mg, about 50 to about 500 mg, about 50 to about 250 mg, about 100 to about 1,000 mg, about 100 to about 500 mg, or about 100 to about 250 mg.

The daily dosage amount of L-threonine will vary depending on a variety of factors. Nonetheless, in various embodiments, the total daily dosage amount of L-threonine is about 10 mg to about 2,000 mg. In further embodiments, the daily dosage amount of metformin is about 10 to about 1,500 mg, about 10 to about 1,000 mg, about 10 to about 500 mg, about 50 to about 2,000 mg, about 50 to about 1,000 mg, about 50 to about 500 mg, about 100 to about 2,000 mg, about 100 to about 1,000 mg, about 100 to about 500 mg, about 250 to about 2,000 mg, about 250 to about 1,500 mg, about 250 to about 1,000 mg, or about 250 to about 500 mg.

The daily dosage amount of the ashwaganda will vary depending on a variety of factors. Nonetheless, in various embodiments, the daily dosage amount of the ashwaganda (root extract, root powder, or combination thereof) is about 10 to about 2,000 mg. In further embodiments, the daily dosage amount of the ashwaganda is about 10 to about 1,500 mg, about 10 to about 1,000 mg, about 10 to about 500 mg, about 25 to about 2,000 mg, about 25 to about 1,000 mg, about 25 to about 500 mg, about 50 to about 2,000 mg, about 50 to about 1,500 mg, about 50 to about 1,000 mg, about 50 to about 500 mg, about 100 to about 2,000 mg, about 100 to about 1,500 mg, about 100 to about 1,000 mg, or about 100 to about 500 mg.

The daily dosage amount of fish oil, krill oil, and/or omega-3-fatty acids will vary depending on a variety of factors. Nonetheless, in various embodiments, the daily dosage amount is from about 5 to about 2,000 mg. In further embodiments, the daily dosage amount of the fish oil, krill oil, and/or omega-3-fatty acids is about 5 to about 1,500 mg, about 5 to about 1,000 mg, about 5 to about 500 mg, about 25 to about 2,000 mg, about 25 to about 1,500 mg, about 25 to about 1,000 mg, about 25 to about 500 mg, about 100 to about 2,000 mg, about 100 to about 1,500 mg, about 100 to about 1,000 mg, about 100 to about 500 mg, about 250 to about 2,000 mg, about 250 to about 1,500 mg, about 250 to about 1,000 mg, about 250 to about 500 mg.

In certain embodiments, the methods described herein (such as a method of administering a composition as described herein to an individual) increase the individual's intracellular levels of reduced glutathione (GSH). The terms “reduced glutathione” and “free glutathione” as used herein are interchangeable.

In some embodiments, the methods described herein elevate the individual's intracellular concentration of reduced glutathione in red blood cells by at least 5 percent, at least 10 percent, about 15 percent, at least 20 percent, at least 25 percent, at least 30 percent, at least 40 percent, or at least about 50 percent, compared to the individual's pre-treatment level of intracellular levels of reduced glutathione in red blood cells.

In various embodiments, administering a composition as described herein to an individual allows the individual to reach an intracellular concentration of reduced glutathione in red blood cells that is from 0.1 mM to 4.0 mM, by about 24 to 48 hours following the first administration of the composition. For example, in certain embodiments an intracellular concentration of reduced glutathione in red blood cells of from 0.5 mM to 3.0 mM is reached by at least about 24 hours after the first administration. In various embodiments, the administration of the composition to an individual is effective in allowing the individual to reach an intracellular concentration of reduced glutathione in red blood cells that is from 2 mM to 4 mM by at least about 72 hours post-first administration. In some embodiments, administering a composition as described herein to an individual results in an intracellular concentration of reduced glutathione in red blood cells being at least 0.3 mM, at least 0.4 mM, at least 0.5 mM, at least 0.6 mM, at least 0.7 mM, at least 0.8 mM, at least 0.9 mM, at least 1.0 mM, at least 1.5 mM, at least 2.0 mM, at least 2.5 mM, at least 3.0 mM, at least 3.5 mM, or that is 4.0 mM, by at least about 72 hours, or by at least about 48 hours, following the first administration of the composition.

Given the increase in glutathione levels that results from administration of the composition of the present disclosure, the composition may be useful in a variety of ways, a number of which are discussed in more detail below.

TB and MRSA

In various embodiments, the methods of instant disclosure are useful for preventing bacterial infection or treating patients suffering from or recovering from a bacterial infection. For example, the compositions and methods of the present disclosure may be used to prevent infection with TB or MRSA in individuals at-risk for infection. Individuals at-risk for infection with tuberculosis or MRSA include, but are not limited to, individuals who come into contact with an individual infected with tuberculosis or MRSA (e.g., health care workers, care givers, etc.). At-risk individuals also include individuals suffering from an autoimmune disease such as human infected with HIV. In addition, immunocompromised individuals are considered at-risk individuals for the purposes of this disclosure. Further nonlimiting examples of individuals at-risk for infection with tuberculosis or MRSA include individuals with one or more medical conditions or factors selected from: older age (for example, age >65 years of age), younger age (for example, <1 year old), obesity or being overweight, pregnancy, chronic kidney disease, diabetes, immunosuppressive disease or receiving immunosuppressive treatment, cardiovascular disease (including congenital heart disease), hypertension, chronic lung diseases (for example, chronic obstructive pulmonary disease, asthma (moderate-to-severe), interstitial lung disease, cystic fibrosis, and pulmonary hypertension), sickle cell disease, neurodevelopmental disorders (for example, cerebral palsy), other conditions that confer medical complexity (for example, genetic or metabolic syndromes and severe congenital anomalies), and having a medical-related technological dependence (for example, tracheostomy, gastrostomy, or positive pressure ventilation). The glutathione support compositions of the present disclosure can be administered to these at-risk individuals prophylactically to prevent or reduce the risk of the individual contracting tuberculosis or MRSA. In various embodiments, the methods of the instant disclosure are particularly useful for individuals at high risk for multidrug resistant tuberculosis.

In still other embodiments, the compositions and methods of the present disclosure may be used to treat an individual infected with TB or MRSA. More specifically, administration of a glutathione support composition of the present disclosure to an individual infected with tuberculosis or MRSA speeds recovery, reduces treatment-related side-effects, helps prevent antibiotic resistance, and/or increases antibiotic efficacy. In various embodiments, the methods of the instant disclosure are particularly useful for individuals suffering from multidrug resistant tuberculosis.

In some embodiments, the glutathione support composition of the present disclosure is administered in conjunction with an antibiotic. The phrase, “in conjunction” does not mean that the glutathione support composition is necessarily formulated together with an antibiotic into a single dosage form (although it can be as discussed previously). In various embodiments, the glutathione support composition is administered separately from administration of an antibiotic treatment for tuberculosis or MRSA. Without being bound by any particular theory, concurrent treatment improves the efficacy of the antibiotic and reduces/mitigates side-effects associated with antibiotic treatment. In other words, concurrent treatment of tuberculosis or MRSA with both an antibiotic and a glutathione support composition of the present disclosure speeds recovery with fewer side effects than treatment with an antibiotic alone. In this aspect, the glutathione support composition may be administered to the individual daily for a consecutive period of time. In one aspect, the glutathione support composition is administered to the individual daily for at least 3 consecutive days during antibiotic treatment. In another aspect, the glutathione support composition is administered daily to the individual for at least 5 consecutive days. In still another aspect, the glutathione support composition is administered to the individual for at least 7 consecutive days. In yet another aspect, the glutathione support composition is administered to the individual for at least 14 consecutive days. In still another aspect, the glutathione support composition is administered to the individual for at least 30 consecutive days. For example, in some embodiments, the glutathione support composition is administered to the individual for at least 60 consecutive days. In other embodiments, the glutathione support composition is administered to the individual for at least 90 consecutive days. In yet other embodiments, daily consecutive dosing of the glutathione support composition is conducted throughout the duration of antibiotic treatment.

In other embodiments, the glutathione support composition may be administered within five days before or after antibiotic treatment begins, for example, the glutathione support compositions can be administered within 4 days, within 3 days, within 2 days, within 1 day, and/or on the same day of the antibiotic treatment begins.

In other embodiments, daily administration of the glutathione support composition begins at least 3 days prior to initiation of antibiotic treatment. In further embodiments, daily administration of the glutathione support compositions begins at least 3 days, at least 4 days, at least 5 days, at least 7 days, at least 8 days, at least 10 days, or at least 14 days prior to initiation of antibiotic treatment, and the daily administration is continued for at least 3 consecutive days, at least 4 consecutive days, at least 5 consecutive days, at least 7 consecutive days, at least 10 consecutive days, at least 14 consecutive days, at least 15 consecutive days, at least 20 consecutive days, at least 25 consecutive days, at least 30 consecutive days, at least 60 consecutive days, or at least 90 consecutive days.

In various embodiments, the glutathione support composition is administered to the individual while the individual with tuberculosis is undergoing antibiotic treatment with one or more antibiotics selected from isoniazid, rifampin, amikacin, aminosalicylic acid, capreomycin, cycloserine, ethambutol, ethionamide, kanamycin, pyrazinamide, rifapentine, rifabutin, streptomycin, ofloxacin, ciprofloxacin, clarithromycin, azithromycin, fluoroquinolones, or a combination thereof.

In various embodiments, the glutathione support composition is administered to the individual with MRSA while the individual is undergoing antibiotic treatment with one or more antibiotics selected from cephapirin, amoxicillin, trimethoprim-sulfonamides, sulfonamides, oxytetracycline, fluoroquinolones, enrofloxacin, danofloxacin, marbofloxacin, cefquinome, ceftiofur, streptomycin, oxytetracycline, vancomycin, cefazolin, cephalothin, cephalexin, linezolid, daptomycin, clindamycin, lincomycin, mupirocin, bacitracin, neomycin, polymyxin B, gentamicin, prulifloxacin, ulifloxacin, fidaxomicin, minocycline, metronidazole, metronidazole, sulfamethoxazole, ampicillin, trimethoprim, ofloxacin, norfloxacin, tinidazole, norfloxacin, ornidazole, levofloxacin, nalidixic acid, ceftriaxone, azithromycin, cefixime, ceftriaxone, cefalexin, ceftriaxone, rifaximin, ciprofloxacin, norfloxacin, ofloxacin, levofloxacin, gatifloxacin, gemifloxacin, prufloxacin, ulifloxacin, moxifloxacin, nystatin, amphotericin B, flucytosine, ketoconazole, posaconazole, clotrimazole, voriconazole, griseofulvin, miconazole nitrate, and fluconazole. In further embodiments, the glutathione support composition is administered to an individual with MRSA while the individual is undergoing treatment with one or more antibiotics selected from trimethoprim-sulfamethoxazole, clindamycin, minocycline, linezolid, or doxycycline.

In various embodiments, the glutathione support composition is administered to an individual with tuberculosis or MRSA while the individual is undergoing antibiotic treatment with an oxazolidinone compound. In this aspect, the oxazolidinone compound may be selected from linezolid, furazolidone, tedizolid, and a combination thereof. In various embodiments, the glutathione support composition is administered to an individual with tuberculosis or MRSA while the individual is undergoing antibiotic treatment with an oxazolidinone compound selected from a compound of Formula (II) or a pharmaceutically acceptable salt thereof:

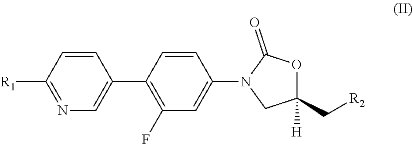

wherein, R₁ is a tetrazole ring substituted at position 2′ with an aminoalkyl, and R₂ is an acetamide. In various embodiments, R₂ is —NHCOCH₃. In further embodiments, R₁ is a tetrazole ring substituted at position 2′ with a di-alkyl-substituted (tertiary) amino group. In further embodiments, R₁ is a tetrazole ring substituted at position 2′ with a dimethyl aminoalkyl or a diethyl aminoalkyl. In yet further embodiments, R₁ is a tetrazole ring substituted at position 2′ with a dimethyl aminoalkyl or a diethyl aminoalkyl; and R₂ is —NHCOCH₃. In other embodiments, R₁ is selected from one of the following:

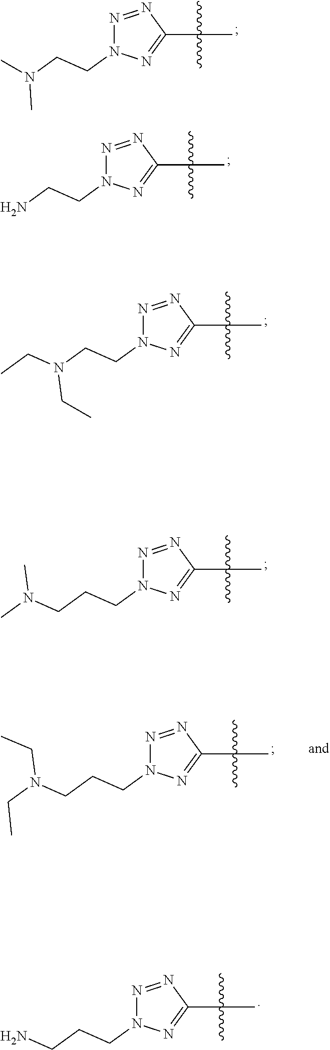 [SMILES: *c1nnn(CCN(C)C)n1 ; *c1nnn(CCN)n1 ; *c1nnn(CCN(CC)CC)n1 ; *c1nnn(CCCN(C)C)n1 ; *c1nnn(CCCN(CC)CC)n1 ; *c1nnn(CCCN)n1]

In various embodiments, the oxazolidinone compound is selected from:

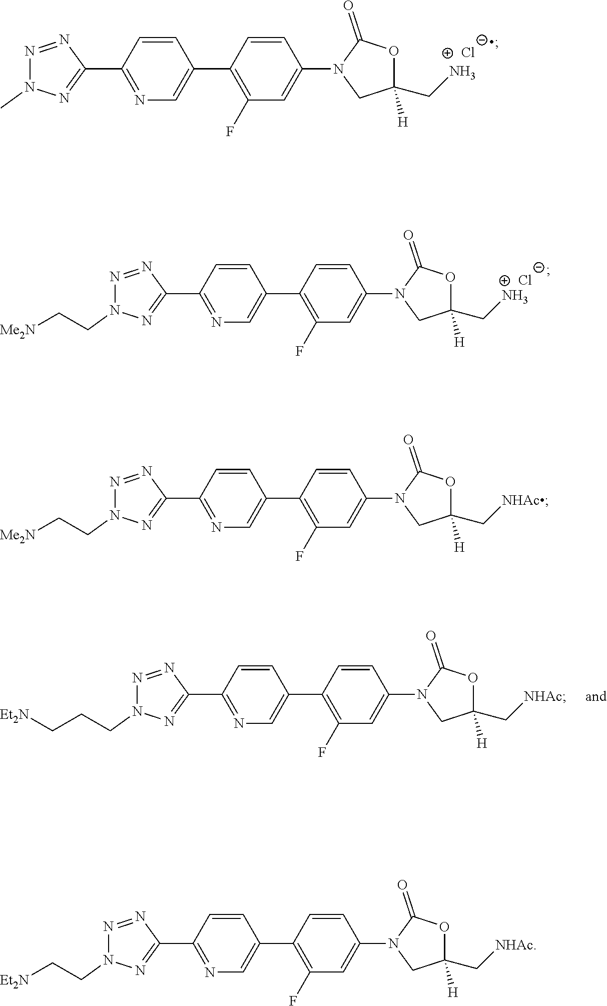

In one embodiment, the oxazolidinone compound is administered in a pharmaceutical composition including liposome vesicles, wherein the liposome vesicles comprise the oxazolidinone antibiotic. For example, in various embodiments, the liposome vesicles include a membrane comprising phosphatidylcholine and cholesterol. In further embodiments, the liposome vesicles include a membrane comprising phosphatidylcholine and cholesterol, wherein the membrane separates inside of the liposome vesicles from an aqueous medium.

Nonlimiting examples phosphatidylcholine include distearoylphosphatidylcholine (DSPC) or hydrogenated soy phosphatidylcholine (HSPC). In various embodiments, the phosphatidylcholine to cholesterol molar ratio is from about 60:40 to 35:6, from about 55:45 to about 35:65, or from about 50:50 to about 40:60. Nonlimiting examples of oxazolidinone compounds and vesicles, such as those described above, appear in US2021/0403463, US2022/0274975, and US2022/0162521, the disclosures of which are incorporated herein by reference in their entirety.

GGT Reduction

In various embodiments, the glutathione support compositions of the instant disclosure are useful in methods for reducing gamma-glutamyltransferase (GGT) levels in the blood. As a result, the liver is better equipped to combat oxidative stress and other forms of damage. The glutathione support compositions decrease GGT levels, thereby resulting in better liver function and quicker self-repair. When the liver is under duress, for example, due to liver disease or damage (e.g., NAFLD or NASH), GGT levels often increase as the liver attempts to compensate for damage. However, when the liver functions normally and produces glutathione, GGT levels can decrease.

Accordingly, the present disclosure relates to methods of decreasing GGT blood levels in an individual including administering to the individual (preferably and animal, mammal, or human) a therapeutically effective amount of the glutathione support compositions of the present disclosure. In various embodiments, the methods reduce GGT levels from baseline by at least 5 percent, wherein baseline is the GGT level before beginning treatment with the glutathione support compositions. In further embodiments, the methods reduce GGT levels by at least 10 percent, at least 15 percent, at least 20 percent, at least 25 percent, about 30 percent, about 35 percent, at least 40 percent, at least 50 percent, at least 60 percent, at least 70 percent, or more (over the GGT level prior to administration of the composition of the present disclosure). For example, administration of the compositions of the present disclosure may reduce GGT levels by about 10 percent to about 75 percent. In one embodiment, the administration of the compositions of the present disclosure reduce GGT levels by about 30 percent to about 80 percent. GGT levels in a subject may be measured by acquiring a blood sample, which is processed to measure the level of GGT in the blood plasma.

In this aspect, since a range of about 9 to about 48 U/L for men and a range of about 7 to about 32 U/L for women is considered typical, administration of the present disclosure may allow for GGT levels of greater than 48 U/L in men or 32 U/L for women to be reduced to a level within the typical ranges. In further embodiments, the methods reduce GGT levels from baseline by at least 2 U/L, wherein baseline is the GGT level before beginning treatment with the glutathione support compositions. In further embodiments, the methods reduce GGT levels from baseline by at least 3 U/L, at least 4 U/L, at least 5 U/L, at least 6 U/L, at least 8 U/L, at least 10 U/L, at least 15 U/L, at least 20 U/L, or more.

In various embodiment, the methods of the instant disclosure are particularly useful for treating an individual having one or more medical conditions or other factors selected from: older age (for example, age >65 years of age), younger age (for example, <1 year old), obesity or being overweight, pregnancy, chronic kidney disease, diabetes, immunosuppressive disease or receiving immunosuppressive treatment, cardiovascular disease (including congenital heart disease), hypertension, chronic lung diseases (for example, chronic obstructive pulmonary disease, asthma (moderate-to-severe), interstitial lung disease, cystic fibrosis, and pulmonary hypertension), bronchiectasis, hemochromatosis, sickle cell disease, neurodevelopmental disorders (for example, cerebral palsy), other conditions that confer medical complexity (for example, genetic or metabolic syndromes and severe congenital anomalies), and having a medical-related technological dependence (for example, tracheostomy, gastrostomy, or positive pressure ventilation).

Vaccine Therapy

The compositions and methods of the present disclosure are also useful for improving vaccine therapy with all types of vaccines because they target the body's response to the vaccine, not necessarily the vaccine per se. In other words, the compositions and methods disclosed herein are widely useful regardless of the type of vaccine because all types of vaccines are similar in their design to prompt an immune response from the body. Vaccines often include carriers, adjuvants such as lipid nanoparticles, polyethylene glycol, PEGylated lipid nanoparticles, graphene oxide, functionalized graphene oxide, polyethylenimine, aluminum, aluminum adjuvants, nano-aluminum adjuvants, aluminum oxyhydroxide-modified graphene oxide nanocomplexes, and liposomes, solvents, preservatives, and the like that are foreign to the body but needed to effectuate the vaccine. An immune response to these types of materials should be targeted or avoided but is often inevitable. Administration of the glutathione support composition during the days or weeks immediately before and/or following administration of a dose of a vaccine increases intracellular glutathione levels thereby also treating or reducing unwanted cellular damage resulting from the body's immune response to the vaccine(s) active (the material designed to cause an immune response to an antigen of a disease) and/or to the additional materials included in the vaccine.

In addition, the compositions and methods of the present disclosure also reduce antibody dependent enhancement and counteract effects of toxic free radicals associated with vaccinations and the body's immune response to the vaccinations, thereby improving patient outcomes. In addition to reducing intracellular damage causes by vaccine therapies, the compositions and methods are also useful for preventing, reducing, and treating side effects associated with vaccine therapies. For example, the compositions and methods of the present disclosure prevent or reduce the likelihood of an individual developing systemic inflammation, hypercoagulation, blood clots, myocarditis, and organ damage. For example, an immune response generates macrophages, which produce free radicals, to fight an infection. While helpful for fighting infection, these free radicals can also cause damage to the host, leading to inflammation. The glutathione support composition may prevent or reduce the inflammation by increasing intracellular glutathione levels and, thus, acting as an important antioxidant to help protect the body from damage to cells caused by free radicals.

The vaccine(s) may be viral vaccines or bacterial vaccines. Nonlimiting examples of viruses that a vaccine may target include viruses of the following families: Coronaviridae, Retroviridae, Arenaviridae, Reoviridae, Rotaviridae, Papillomaviridae, Influenza, Adenoviridae, Flaviviridae, Herpesviridae, Filoviridae, Pneumoviridae, Orthomyxoviridae, and Ebola virus (Ebola Virus Disease (EVD)).

In various embodiments, the compositions and methods of the present disclosure are particularly well-suited for improving vaccine therapies using mRNA vaccines. For example, the glutathione support composition is particularly useful for improving vaccine therapies with vaccines and mRNA vaccines using nanoparticle-based materials and vaccines comprising adjuvants such graphene derivatives, for example graphene oxide. Furthermore, in some embodiments, the compositions and methods are useful for improving vaccine therapies for viruses of the family Coronaviridae (for example, COVID-19), Retroviridae (for example, HIV), Papillomaviruses (for example, human papillomaviruses), and varicella zoster virus (VZV). Furthermore, in other embodiments, the compositions and methods are useful for improving vaccine therapies for Ebola virus.

Other Uses

The glutathione support compositions of the present disclosure are useful for treating viral infections, for example, COVID-19, influenza, the common cold, etc., as well as fungal infections. Treatment with the glutathione support compositions show a decrease in high-sensitivity C-reactive protein (hs-CRP) levels, improved complete blood count (CBC) levels, decrease in homocysteine levels and/or improved percent iron saturation. Individuals treated with the glutathione support compositions show reduced symptoms and a shortened time to clinical recovery, compared with individuals not treated with the glutathione support compositions.

In various embodiments, the glutathione support composition may be administered to improve respiratory function, for example, in individuals suffering from bronchiectasis. In other embodiments, the glutathione support composition may be used to reduce or prevent the likelihood of contracting a viral infection, including respiratory viral infections. In further embodiments, the glutathione support composition may be used to treat viral infection, including respiratory viral infections. In still other embodiments, the glutathione support composition may be used to reduce viral load in an individual. In yet other embodiments, the glutathione support composition may be used to prevent and/or treat viruses of the family Coronaviridae (for example, COVID-19), Retroviridae (for example, HIV), Papillomaviruses (for example, human papillomaviruses), Flaviviridae (for example, Zika), and varicella zoster virus (VZV). Furthermore, in other embodiments, the compositions and methods are useful for treating Ebola virus.

In still other embodiments, the glutathione support compositions are useful in methods for modulating, stabilizing, or normalizing metabolic function. In other embodiments, glutathione support compositions are useful in methods for improving bone health. In yet other embodiments, the glutathione support compositions are useful in methods for increase the production of Vitamin D.

Without wishing to be bound by any particular theory, it is also believed that the glutathione support compositions of the present disclosure reduce cortisol levels, especially in individual suffering from sleep deprivation, stress, physical exertion, and the like. Reducing cortisol levels is useful for treating sleep disorders, improving immunity, and reducing the risk of long-term health problems associated with high cortisol levels, for example, hypertension, stroke, obesity, inflammatory diseases (e.g., Crohns disease), and the like.

In various embodiments, the glutathione support compositions are useful for modulating, stabilizing, or normalizing hormone levels, for example, levels of estrogen or testosterone. This may be achieved by increasing sex hormone-binding globulin (SHBG), a protein that binds to sex hormones in the bloodstream. SHBG helps regulate estrogen and testosterone in the body. By binding to these hormones, it can reduce the amount of free testosterone and estrogen, helping to maintain a healthy hormone balance. High levels of SHBG have been associated with better bone mineral density in both men and women. Additionally, higher levels of SHBG are linked to improved insulin sensitivity and can help regulate blood sugar levels and support healthy metabolism.

In various embodiments, the glutathione support compositions are particularly useful for individuals having one or more comorbidities. Furthermore, in various embodiments, the methods of the instant disclosure are particularly useful for treating an at-risk individual (such as those described above with respect to individuals at risk for TB and/or MRSA.

### EXAMPLES

The following non-limiting example is merely illustrative of the embodiments of the present invention, and are not to be construed as limiting the invention, the scope of which is defined by the appended claims.

### Example 1: Formulation Examples

Table 1 below provides glutathione support compositions made in accordance with the present disclosure.

TABLE 1
Glutathione Support Compositions

| | A | | B | | C | |
|---|---|---|---|---|---|---|
| | Dosage | Weight | Dosage | Weight | Dosage | Weight |
| Component | (mg) | Percent | (mg) | Percent | (mg) | Percent |
| (a) Collagen | 1299 | 52 | 1099 | 44 | 1099 | 44 |
| Source | | | | | | |
| (b) Glutamate | 800 | 32 | 1000 | 40 | 700 | 28 |
| Source | | | | | | |
| (c) Cysteine Source | 400 | 16 | 400 | 16 | 700 | 28 |
| Ratio of (b):(c) | 2:1 | 2:1 | 2.5:1 | 2.5:1 | 1:1 | 1:1 |
| Ratio of (a):((b) + (c)) | 1.1:1 | 1.1:1 | 0.8:1 | 0.8:1 | 0.8:1 | 0.8:1 |
| (d) Selenium | 0.01 (10 | 0.01 | 0.01 (10 | 0.01 | 0.01 (10 | 0.01 |
| Source | mcg) | (40 | mcg | (40 | mcg) | (40 |
| | | ppm) | | ppm) | | ppm) |
| (e) Boron Source | 0.75 | 0.03 | 0.75 | 0.03 | 0.75 | 0.03 |
| | | (35 | | (35 | | (35 |
| | | ppm) | | ppm) | | ppm) |
| Total | 2500 mg | 100 | 2500 mg | 100 | 2500 mg | 100 |

Any of the collagen sources, glutamate sources, cysteine sources, selenium sources, and boron sources discussed previously may be used in the formulations in Table 1. By way of example, in one embodiment, Composition A included marine collagen peptides as the collagen source, L-glutamine as a glutamate source, L-cystine as the cysteine source, selenomethionine as the selenium source, and boron salt as the boron source. In another embodiment, Composition A included ovine collagen peptides as the collagen source, L-glutamine as a glutamate source, L-cystine as the cysteine source, selenomethionine as the selenium source, and boron salt as the boron source. In yet another embodiment, Composition A included marine collagen peptides as the collagen source, N-Acetylglutamate as a glutamate source, L-cystine as the cysteine source, selenomethionine as the selenium source, and boron salt as the boron source. In still another embodiment, Composition A included ovine collagen peptides as the collagen source, N-Acetylglutamate as a glutamate source, L-cystine as the cysteine source, selenomethionine as the selenium source, and boron salt as the boron source. In another embodiment, Composition A included marine collagen peptides as the collagen source, N-Acetylglutamate as a glutamate source, N-Acetyl-Cysteine as the cysteine source, selenomethionine as the selenium source, and boron salt as the boron source. In yet another embodiment, Composition A included ovine collagen peptides as the collagen source, N-Acetylglutamate as a glutamate source, N-Acetyl-Cysteine as the cysteine source, selenomethionine as the selenium source, and boron salt as the boron source. In another embodiment, Composition A included marine collagen peptides as the collagen source, L-glutamine as a glutamate source, N-Acetyl-Cysteine as the cysteine source, selenomethionine as the selenium source, and boron salt as the boron source. In yet another embodiment, Composition A included ovine collagen peptides as the collagen source, L-glutamine as a glutamate source, N-Acetyl-Cysteine as the cysteine source, selenomethionine as the selenium source, and boron salt as the boron source.

In one embodiment, Composition B included marine collagen peptides as the collagen source, N-Acetylglutamate as a glutamate source, L-cystine as the cysteine source, selenomethionine as the selenium source, and boron salt as the boron source. In another embodiment, Composition B included ovine collagen peptides as the collagen source, N-Acetylglutamate as a glutamate source, L-cystine as the cysteine source, selenomethionine as the selenium source, and boron salt as the boron source. In yet another embodiment, Composition B included marine collagen peptides as the collagen source, L-glutamine as a glutamate source, L-cystine as the cysteine source, selenomethionine as the selenium source, and boron salt as the boron source. In another embodiment, Composition B included ovine collagen peptides as the collagen source, L-glutamine as a glutamate source, L-cystine as the cysteine source, selenomethionine as the selenium source, and boron salt as the boron source. In another embodiment, Composition B included marine collagen peptides as the collagen source, L-glutamine as a glutamate source, N-Acetyl-Cysteine as the cysteine source, selenomethionine as the selenium source, and boron salt as the boron source. In still another embodiment, Composition B included ovine collagen peptides as the collagen source, L-glutamine as a glutamate source, N-Acetyl-Cysteine as the cysteine source, selenomethionine as the selenium source, and boron salt as the boron source. In yet another embodiment, Composition B included marine collagen peptides as the collagen source, N-Acetylglutamate as a glutamate source, N-Acetyl-Cysteine as the cysteine source, selenomethionine as the selenium source, and boron salt as the boron source. In still another embodiment, Composition B included ovine collagen peptides as the collagen source, N-Acetylglutamate as a glutamate source, N-Acetyl-Cysteine as the cysteine source, selenomethionine as the selenium source, and boron salt as the boron source.

In one embodiment, Composition C included marine collagen peptides as the collagen source, N-Acetylglutamate as a glutamate source, N-Acetyl-Cysteine as the cysteine source, selenomethionine as the selenium source, and boron salt as the boron source. In another embodiment, Composition C included ovine collagen peptides as the collagen source, N-Acetylglutamate as a glutamate source, N-Acetyl-Cysteine as the cysteine source, selenomethionine as the selenium source, and boron salt as the boron source. In yet another embodiment, Composition C included marine collagen peptides as the collagen source, L-glutamine as a glutamate source, N-Acetyl-Cysteine as the cysteine source, selenomethionine as the selenium source, and boron salt as the boron source. In still another embodiment, Composition C included ovine collagen peptides as the collagen source, L-glutamine as a glutamate source, N-Acetyl-Cysteine as the cysteine source, selenomethionine as the selenium source, and boron salt as the boron source. In another embodiment, Composition C included marine collagen peptides as the collagen source, N-Acetylglutamate as a glutamate source, L-cystine as the cysteine source, selenomethionine as the selenium source, and boron salt as the boron source. In still another embodiment, Composition C included ovine collagen peptides as the collagen source, N-Acetylglutamate as a glutamate source, L-cystine as the cysteine source, selenomethionine as the selenium source, and boron salt as the boron source. In another embodiment, Composition C included marine collagen peptides as the collagen source, L-glutamine as a glutamate source, L-cystine as the cysteine source, selenomethionine as the selenium source, and boron salt as the boron source. In still another embodiment, Composition C included ovine collagen peptides as the collagen source, L-glutamine as a glutamate source, L-cystine as the cysteine source, selenomethionine as the selenium source, and boron salt as the boron source.

### Example 2: GGT and Hs-CRP Levels

Gamma-glutamyltransferase (GGT) and high-sensitivity C-reactive protein (hs-CRP) levels were monitored in four subjects prior to administration of a glutathione support composition of the present disclosure and at periodic intervals after administration of a daily dose. All subjects received Composition A-1 in Table 2.

TABLE 2
Glutathione Support Composition

| | | A-1 | |
|---|---|---|---|
| | | Dosage | Weight |
| Component | | (mg) | Percent |
| (a) Collagen Source | Marine Collagen | 1299 | 52 |
| | Peptides | | |
| (b) Glutamate Source | L-Glutamine | 800 | 32 |
| | N- | | |
| | Acetylglutamate | | |
| (c) Cysteine Source | L-Cystine | 400 | 16 |
| | N-Acetyl- | | |
| | Cysteine | | |
| Ratio of (b):(c) | | 2:1 | 2:1 |
| Ratio of (a):((b) + (c)) | | 1.1:1 | 1.1:1 |
| (d) Selenium Source | Selenomethionine | 0.01 | 0.01 |
| | | (10 mcg) | (40 ppm) |
| (e) Boron | Boron Salt | 0.75 | 0.03 |
| | | | (35 ppm) |
| | Total | 2500 mg | 100 |

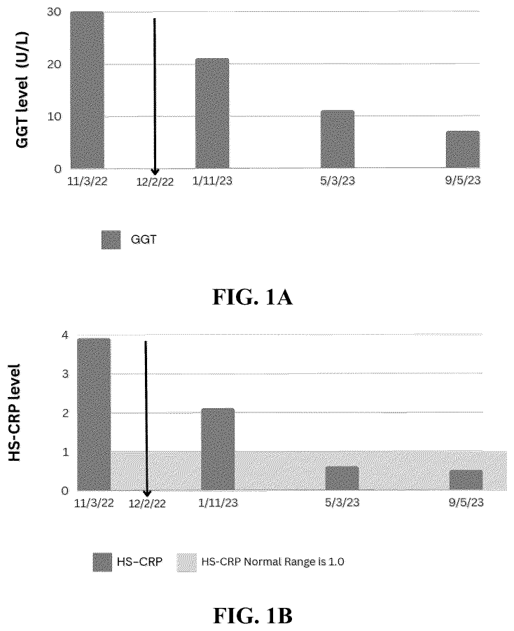

The particulars of dosing regimen and change in GGT and hs-CRP levels from the blood draw prior to administration and final blood draw post-administration are provided below in Table 3 below. The graphical representation of the GGT and hs-CRP levels for each subject are provided in FIGS. 1A-4B where the arrow indicates the beginning of administration of Composition A-1 to each subject.

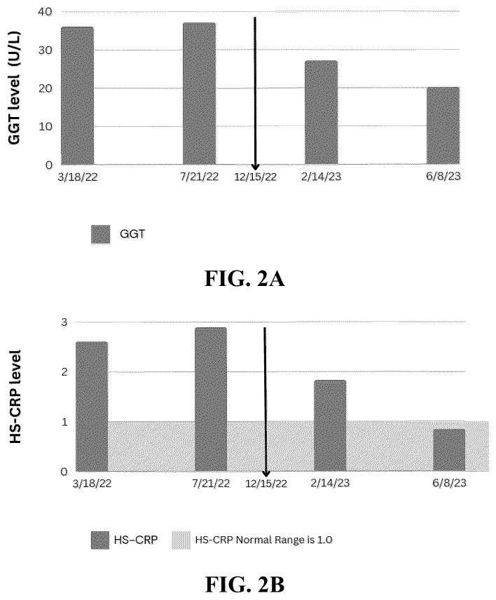

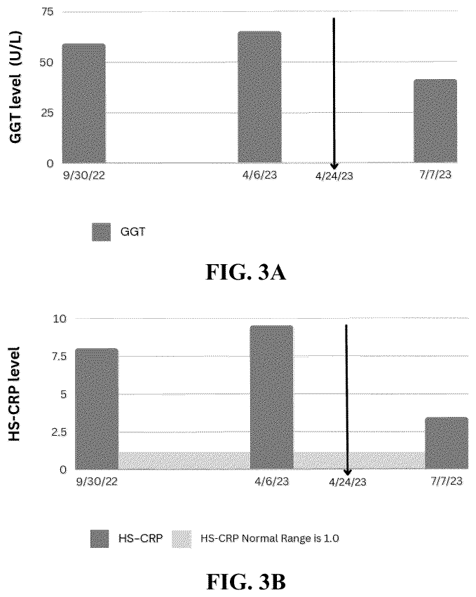

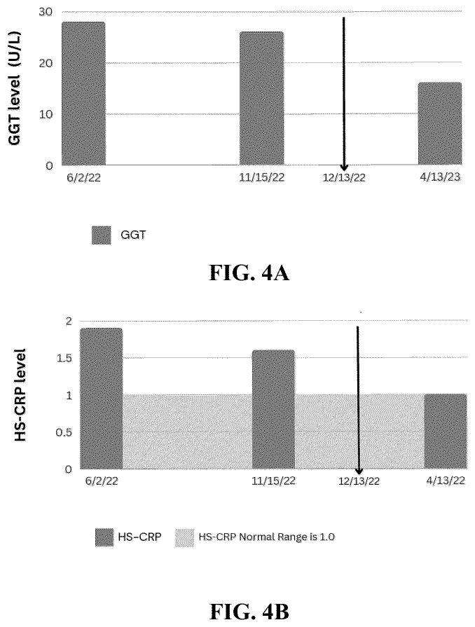

TABLE 3
Clinical Case Studies

| | Subject | 1 | 2 | 3 | 4 |
|---|---|---|---|---|---|
| | Composition | A | A | A | A |
| | Dosing Regimen (per | 2.5 grams | 2.5 grams | 2.5 grams | 2.5 grams 2× |
| | day) | | | | for 5 weeks; |
| | | | | | 2.5 grams 1× |
| | | | | | thereafter |
| GGT | Levels | FIG. 1A | FIG. 2A | FIG. 3A | FIG. 4A |
| | Δ_{Pre Dosing-Final Blood Draw} | 23 | 19 | 24 | 10 |
| hs-CRP | Levels | FIG. 1B | FIG. 2B | FIG. 3B | FIG. 4B |
| | Δ_{Pre Dosing-Final Blood Draw} | 3.4 | 1.77 | 6.6 | 0.6 |

As evident from Table 3 and FIGS. 1A, 2A, 3A, and 4A, GGT levels dropped by at least 10 U/L in subjects receiving Composition A. All subjects receiving Composition A also experienced a reduction in hs-CRP levels.

### Example 3: Procedure for Assessing Glutathione Levels

Glutathione (GSH) levels can be ascertained using a Glutathione Colorimetric Detection Kit, for example, from ImmunoChemistry Technologies, LLC (Bloomington, MN), KIT #9135, which is designed to quantitatively measure glutathione (GSH) and oxidized glutathione (GSSG) present in a variety of samples such as whole blood; serum; plasma; erythrocytes; urine; cell lysates; and tissue samples. A GSSG standard is used to generate a standard curve for the assay. A reaction mixture containing NADPH and Glutathione Reductase converts the oxidized dimer, GSSG, into Free GSH (1 GSSG=2 GSH). A Colorimetric GSH Detection Substrate for reaction with the free thiol group on GSH to yield a highly colored product. Reagents are in solution and require simple dilution for use in the assay. By using 2-Vinylpyridine (2VP) to block any Free GSH in the sample, the concentration of Oxidized Glutathione (GSSG) can be determined. Any samples that have not been treated with 2VP will yield Total GSH levels (Free GSH and GSSG). The Free GSH concentration in the sample is calculated from the difference between the Total GSH determined and the GSH generated from GSSG for the 2VP-treated samples. The concentration of GSH can be determined either as an endpoint read of the color developed at 405 nm or by measuring the rate of color development at 405 nm.

The assay can be analyzed with detection equipment, for example, with a plate reader, a 96-well plate reader capable of reading optical absorption at 405-412 nm, and software for converting raw optical density readings from the plate reader and conducting 4-parameter logistic curve (4PLC) fitting. Glutathione concentrations are calculated from the data using the curve fitting routine supplied with the plate reader. Oxidized Glutathione (GSSG) concentrations of the samples are determined from the data obtained from 2VP-treated samples read off the 2VP-treated standard curve. The concentration of GSSG in the samples would be half of the GSH concentration read off the curve.

1 GSSG=2 GSH

Free glutathione (GSH) concentrations are obtained by subtracting the Oxidized Glutathione (GSSG) levels obtained from the 2VP-treated standard and samples from non-treated standards and samples (Total GSH). Concentrations obtained are in pM of Glutathione.

Total GSH=Free GSH+Oxidized GSH (GSSG)

Oxidized GSH=(Concentration of 2VP-treated GSH)/2

Free GSH=Total GSH Concentration−Oxidized GSH Concentration

Linearity is determined by taking Jurkat cell lysates at 25×106 cells/mL and one at 1.28×106 cells/mL, diluted 1:5, and mixed in the ratios given below. The measured concentrations are compared to the expected values based on ratios used.
Reported ranges for glutathione levels are set forth in Table 4.

TABLE 4
Reported Normal Ranges for Glutathione

| Reference | Sample | Total GSH | Free GSH | GSSG |
|---|---|---|---|---|
| Clinical Chemistry, 48/5: 742-53 | Whole Blood | 914-996 ± | 756-945 ± | 5.66-6.6 ± |
| (2002) | | 101 μM | 95 μM | 155 μM |
| Internet Journal of Alternative | EDTA | | 95.2 ± | |
| Medicine 2/1 (2005) | Erythrocytes | | 11.5 μM | |
| Diabetes, 48: 1850-55 (1999) | Erythrocytes | | 5.7 ± | |
| | | | 0.3 μM | |
| Clinical Chemistry, 48/5: 742-53 | Erythrocytes | | | 3.27 ± |
| (2002) | | | | 1.15 μM |
| J. Biol. Chem., 83/2: 361-65 (1929) | EDTA | 7.3 ± | 3.4 ± | |
| | Plasma | 1.8 μM | 0.9 μM | |
| Clinical Chemistry, 33/9: 1675-76 | Plasma | 3.35 ± | | 0.35 ± |
| (1987_{—} | | 1.07 μM | | 0.14 μM |
| J. Biol. Chem., 282/19: 14337-4 (2007) | Jurkat Lysate | | 40 ng/mg | |
| | | | total protein | |
| FASEB J., 14: 1352-61 (2000) | Jurkat Lysate | | 15 ng/mg | |
| | | | total Protein | |

The protocol discussed above is based on the following references, which are incorporated herein by reference in their entirety:
Meister, A. 1998. On the Discovery of Glutathione, Trends Biochem. Sci. 13(5): 185-188.
Meister, A. 1994. The Glutathione Ascorbic Acid Antioxidant Systems in Animals, J. Biol. Chem. 269:9397-9400.
Drôge W., et al., 1994. Functions of Glutathione and Glutathione Disulfide in Immunology and Immuno-pathology, FASEB J., 8:1131-1138.

The compositions and methods of the present disclosure can comprise, consist of, or consist essentially of the essential elements and limitations of the disclosure described herein, as well as any additional or optional ingredients, components, or limitations described herein or otherwise useful. The terms “comprising,” “having,” and “including” are used in their open, non-limiting sense. The terms “a” and “the” are understood to encompass the plural as well as the singular. “Pharmaceutically acceptable salt” and “nutraceutical acceptable salt” refer to salts of the compounds of the glutathione support compositions derived from the combination of such compounds and an organic or inorganic acid (acid addition salts) or an organic or inorganic base (base addition salts). The compounds of the glutathione support compositions may be used in either the free base or salt forms, with both forms being considered as being within the scope of the present disclosure. Some of the compounds discussed throughout the disclosure may be in the form of a salt in the composition or added to the composition in the form of a salt (and dissociate in the composition). Thus, all compounds and amounts of compounds relate to both the salt form of the compound and to the disassociated form of the compound. Notwithstanding the phrase, “a salt thereof,” used in conjunction with a compound or ingredient, it is understood that the salt form of the compound or ingredient is included, unless expressly stated otherwise. In other words, it is understood that salt forms are included even without specifically or expressly stating the compound or ingredient can form salts or can be available as salts. The salts referred to throughout the disclosure may include, without limitation, salts having a counter-ion such as an alkali metal, alkaline earth metal, or ammonium counter-ion. This list of counter-ions, however, is non-limiting.

All percentages, parts and ratios herein are based upon the total weight of the compositions of the present disclosure, unless otherwise indicated. All ranges and values disclosed herein are inclusive and combinable. The expression “inclusive” for a range of concentrations means that the limits of the range are included in the defined interval. For example, any value or point described herein that falls within a range described herein can serve as a minimum or maximum value to derive a sub-range, etc. Furthermore, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Other than in the operating examples, or where otherwise indicated, all numbers expressing quantities of ingredients and/or reaction conditions are to be understood as being modified in all instances by the term “about.” As used herein, the expression “at least one” is interchangeable with the expression “one or more” and thus includes individual components as well as mixtures/combinations.

The term “active material” as used herein with respect to the percent amount of an ingredient or raw material, refers to 100 percent activity of the ingredient or raw material.

Throughout the disclosure, the term “a combination thereof” (or a mixture thereof) may be used following a list of elements as shown in the following example where letters A-F represent the elements: “one or more elements selected from the group consisting of A, B, C, D, E, F, and a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from the group consisting of A, B, C, D, E, F, and a combination of any two or more of A, B, C, D, E, and F.”

All components and elements positively set forth in this disclosure can be negatively excluded from the claims. In other words, the compositions of the instant disclosure can be free or essentially free of all components and elements positively recited throughout the instant disclosure. The term “essentially free” as used herein means that there is less than about 5 percent by weight of a specific material added to a composition, based on the total weight of the compositions. Nonetheless, the compositions may include less than about 2 weight percent, less than about 1 weight percent, less than about 0.5 weight percent, less than about 0.1 weight percent, less than 0.01 weight percent, or none of the specified material.

Some of the various categories of components identified may overlap. In such cases where overlap may exist and the composition includes both components (or the composition includes more than two components that overlap), an overlapping component or ingredient does not represent more than one component. In other words, a single compound, ingredient, or step cannot simultaneously serve as two different components, ingredients, or step of a claimed compositions and methods.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A glutathione support composition for increasing intracellular glutathione levels in a subject, comprising:
(a) a collagen source, wherein the collagen source comprises proteins or peptides comprising at least 5 percent proline residues, at least 5 percent hydroxyproline residues, and at least 20 percent glycine residues based on a total weight average of the proteins or peptides;
(b) a glutamate source;
(c) a cysteine source; and
(d) a selenium source.

2. The glutathione support composition of claim 1, further comprising a boron source.

3. The glutathione support composition of claim 1, further comprising one or more active ingredients.

4. The glutathione support composition of claim 3, wherein the one or more active ingredients is selected from the group consisting of a zinc source, methylfolate, methyl-B₁₂, quercetin, sulforaphane, methionine, vitamin D, vitamin C, L-threonine, L-theanine, turmeric, curcumin, taurine, melatonin, ashwagandha, fisetin, 1-NMA, NAD+, or kaempferol (or other flavonoid antioxidants), and combinations thereof.

5. The glutathione support composition of claim 1, wherein the collagen source is present in an amount of about 30 to about 60 weight percent, the glutamate source is present in an amount of about 20 to about 50 weight percent, and the cysteine source is present in an amount of about 10 to about 30 weight percent, based on the total weight of the glutathione support composition.

6. The glutathione support composition of claim 1, wherein the collagen source and the glutamate and cysteine sources are in present in an ratio of 1:3 to 3:1 ((a):(b)+(c)).

7. The glutathione support composition of claim 1, wherein the glycine, glutamate, and cysteine sources ((a)+(b)+(c)) constitute about 50 to about 99.99 weight percent based on the total weight of the glutathione support composition.

8. The glutathione support composition of claim 1, wherein the collagen source is selected from the group consisting of collagen, collagen peptides, gelatin, gelatin peptides, elastin, elastin peptides, glycine-rich RNA-binding proteins, tenascin-C, proteoglycans, hydrolysates thereof (peptides thereof), and combinations thereof.

9. The glutathione support composition of claim 1, wherein the collagen source comprises collagen peptides, gelatin peptides, elastin peptides, or a combination thereof.

10. The glutathione support composition of claim 1, wherein the cysteine source comprises L-cystine, N-acetyl cysteine, a salt thereof, or a combination thereof.

11. The glutathione support composition of claim 1, wherein the glutamate source comprises L-glutamine, a L-glutamine precursor, glutamic acid, a glutamic acid precursor, a salt thereof, or a combination thereof.

12. The glutathione support composition of claim 1, wherein the selenium source is selected from selenomethionine, selenocysteine, selenite, methyl selenocysteine, selenium nanoparticles, selenocysteine, Se-methylselenocysteine, and a combination thereof. In a preferred embodiment, the selenium source is selected from selenomethionine, methylselenocysteine, and combinations thereof.

13. A glutathione support composition for increasing intracellular glutathione levels in a subject, comprising:
(a) a collagen source present in an amount of about 35 to about 75 weight percent based on the total weight of the composition, wherein the collagen source comprises hydrolyzed collagen, hydrolyzed gelatin, hydrolyzed elastin, or a mixture thereof;
(b) a glutamate source present in an amount of about 20 to about 50 weight percent based on the total weight of the composition, wherein the glutamate source comprises L-glutamine, a L-glutamine precursor, a salt thereof, or a combination thereof;
(c) a cysteine source present in an amount of about 10 to about 30 weight percent based on the total weight of the composition, wherein the cysteine source comprises L-cystine, a L-cysteine precursor other than L-cystine, a salt thereof, or a combination thereof; and
(d) a selenium source.

14. The glutathione support composition of claim 13, wherein the selenium source is present in an amount such that the glutathione support composition comprises about 50 to about 300 ppm of elemental selenium.

15. The glutathione support composition of claim 13, further comprising a boron source.

16. The glutathione support composition of claim 15, wherein the boron source is present in an amount such that the glutathione support composition comprises about 20 to about 400 ppm of elemental boron.

17. The glutathione support composition of claim 13, further comprising taurine, melatonin, sulforaphane, quercetin, Vitamin D3, Vitamin k2, Vitamin C, metformin, L-threonine, L-theanine, turmeric, curcumin, ashwagandha or an extract thereof, fish oil, krill oil, omega 3 fatty acids, odd-chain fatty acids, botanical extracts, or a combination thereof.

18. A method of increasing intracellular glutathione in a subject, comprising:
administering a glutathione support composition to the subject in a therapeutically effective amount, wherein the glutathione support composition comprises:
(a) a collagen source, wherein the collagen source comprises proteins or peptides comprising at least 5 percent proline residues, at least 5 percent hydroxyproline residues, and at least 20 percent glycine residues based on a total weight average of the proteins or peptides;
(b) a glutamate source;
(c) a cysteine source;
(d) a selenium source; and
(e) a boron source.

19. The method of claim 18, wherein the step of administering comprises providing the subject the therapeutically effective amount in a daily dosage, wherein the daily dosage is administered to the individual for a dosing period of at least 3 consecutive days, and wherein the subject has a ratio of intracellular reduced glutathione (GSH) to oxidized gluthathione (GSSH) of at least 5:1 after the dosing period.

20. The method of claim 18, wherein the collagen source is present in an amount of about 35 to about 75 weight percent based on the total weight of the glutathione support composition, and wherein the collagen source comprises hydrolyzed marine or ovine collagen, hydrolyzed marine or ovine gelatin, hydrolyzed elastin, or a mixture thereof.
